SEQUENCE LISTING (9 sequences)

SEQ ID NO: 1 - protein, 242 aa, Lycopersicon esculentum
MGRGKVELKRIENKINRQVTFAKRRNGLLKKAYELSILCDAEIALIIFSSRGKLYEFCSNSSMSKTLERYHRYNYGTLEGTQTSSDSQNNYQEYLKLKTRVEMLQQSQRHLLGEDLGQLGTKDLEQLERQLDSSLRQIRSTKTQHILDQLAELQQKEQSLTEMNKSLRIKLEELGVTFQTSWHCGEQSVQYRHEQPSHHEGFFQHVNCNNTLPISYGYDNVQPENAAPSTHDATGVVPGWML

SEQ ID NO: 2 - protein, 92 aa, Lycopersicon esculentum
MGRGKVELKRIENKINRQVTFAKRRNGLLKKAYELSILCDAEIALIIFSSRGKLYEFCSNSSMSKTLERYHRYNYGTLEGTQTSSDSQAFAR

SEQ ID NO: 3 - protein, 242 aa, Lycopersicon esculentum
MGRGKVELKRIENKINRQVTFAKRRNGLLKKAYELSILCDAEIALIIFSSRGKLYEFCSNSSMSKTLERYHRYNYGTLEGTQTSSDSQNNYQEYLKLKTRVKMLQQSQRHLLGEDLGQLGTKDLEQLERQLDSSLRQIRSTKTQHILDQLAELQQKEQSLTEMNKSLRIKLEELGVTFQTSWHCGEQSVQYRHEQPSHHEGFFQHVNCNNTLPISYGYDNVQPENAAPSTHDATGVVPGWML

SEQ ID NO: 4 - protein, 242 aa, Lycopersicon esculentum
MGRGKVELKRIENKINRQVTFAKRRNGLLKKAYELSILCDAEIALIIFSSRGKLYEFCSNSSMSKTLERYHRYNYGTLEGTQTSSDSQNNYQEYLKLKTRVEMLQQSQRHLPGEDLGQLGTKDLEQLERQLDSSLRQIRSTKTQHILDQLAELQQKEQSLTEMNKSLRIKLEELGVTFQTSWHCGEQSVQYRHEQPSHHEGFFQHVNCNNTLPISYGYDNVQPENAAPSTHDATGVVPGWML

SEQ ID NO: 5 - DNA, 729 bp, Lycopersicon esculentum
atgggtagagggaaagtagaattgaagagaattgagaacaaaataaatagacaagttacctttgcaaagagaagaaatggactcctaaagaaagcttatgaactttctatactttgtgatgctgaaattgctcttattattttctctagtcgtggcaagctttatgaattttgcagcaattcaagtatgtccaagacattggagagataccacagatacaattatggtacacttgaaggaacccaaacttcatcagattcacagaacaactaccaagagtatttgaagcttaaaacaagagtggaaatgttacaacagtctcaaaggcatttgctaggtgaggatttgggacaattgggcacaaaagacttggaacagcttgaacgtcaattggattcatcattgaggcaaattaggtcaacaaagacacaacacattcttgatcaacttgctgaacttcaacaaaaggaacaatctcttactgaaatgaacaaatctttgagaataaagttggaagaacttggtgttacctttcaaacatcatggcattgtggtgagcaaagtgtacaatatagacatgaacagccttctcatcatgagggattttttcaacatgtaaattgcaataatacattgcctataagttacggatacgataatgtacaacccgaaaatgcagcaccatcaacacatgatgctactggagttgtacctggatggatgctttga

SEQ ID NO: 6 - DNA, 667 bp, Lycopersicon esculentum
atgggtagagggaaagtagaattgaagagaattgagaacaaaataaatagacaagttacctttgcaaagagaagaaatggactcctaaagaaagcttatgaactttctatactttgtgatgctgaaattgctcttattattttctctagtcgtggcaagctttatgaattttgcagcaattcaagtatgtccaagacattggagagataccacagatacaattatggtacacttgaaggaacccaaacttcatcagattcacaggcatttgctaggtgaggatttgggacaattgggcacaaaagacttggaacagcttgaacgtcaattggattcatcattgaggcaaattaggtcaacaaagacacaacacattcttgatcaacttgctgaacttcaacaaaaggaacaatctcttactgaaatgaacaaatctttgagaataaagttggaagaacttggtgttacctttcaaacatcatggcattgtggtgagcaaagtgtacaatatagacatgaacagccttctcatcatgagggattttttcaacatgtaaattgcaataatacattgcctataagttacggatacgataatgtacaacccgaaaatgcagcaccatcaacacatgatgctactggagttgtacctggatggatgctttga

SEQ ID NO: 7 - DNA, 729 bp, Lycopersicon esculentum
atgggtagagggaaagtagaattgaagagaattgagaacaaaataaatagacaagttacctttgcaaagagaagaaatggactcctaaagaaagcttatgaactttctatactttgtgatgctgaaattgctcttattattttctctagtcgtggcaagctttatgaattttgcagcaattcaagtatgtccaagacattggagagataccacagatacaattatggtacacttgaaggaacccaaacttcatcagattcacagaacaactaccaagagtatttgaagcttaaaacaagagtgaaaatgttacaacagtctcaaaggcatttgctaggtgaggatttgggacaattgggcacaaaagacttggaacagcttgaacgtcaattggattcatcattgaggcaaattaggtcaacaaagacacaacacattcttgatcaacttgctgaacttcaacaaaaggaacaatctcttactgaaatgaacaaatctttgagaataaagttggaagaacttggtgttacctttcaaacatcatggcattgtggtgagcaaagtgtacaatatagacatgaacagccttctcatcatgagggattttttcaacatgtaaattgcaataatacattgcctataagttacggatacgataatgtacaacccgaaaatgcagcaccatcaacacatgatgctactggagttgtacctggatggatgctttga

SEQ ID NO: 8 - DNA, 729 bp, Lycopersicon esculentum
atgggtagagggaaagtagaattgaagagaattgagaacaaaataaatagacaagttacctttgcaaagagaagaaatggactcctaaagaaagcttatgaactttctatactttgtgatgctgaaattgctcttattattttctctagtcgtggcaagctttatgaattttgcagcaattcaagtatgtccaagacattggagagataccacagatacaattatggtacacttgaaggaacccaaacttcatcagattcacagaacaactaccaagagtatttgaagcttaaaacaagagtggaaatgttacaacagtctcaaaggcatttgccaggtgaggatttgggacaattgggcacaaaagacttggaacagcttgaacgtcaattggattcatcattgaggcaaattaggtcaacaaagacacaacacattcttgatcaacttgctgaacttcaacaaaaggaacaatctcttactgaaatgaacaaatctttgagaataaagttggaagaacttggtgttacctttcaaacatcatggcattgtggtgagcaaagtgtacaatatagacatgaacagccttctcatcatgagggattttttcaacatgtaaattgcaataatacattgcctataagttacggatacgataatgtacaacccgaaaatgcagcaccatcaacacatgatgctactggagttgtacctggatggatgctttga

SEQ ID NO: 9 - DNA, 5286 bp, Lycopersicon esculentum
  exon (1)..(185): exon 1
atgggtagagggaaagtagaattgaagagaattgagaacaaaataaatagacaagttacctttgcaaagagaagaaatggactcctaaagaaagcttatgaactttctatactttgtgatgctgaaattgctcttattattttctctagtcgtggcaagctttatgaattttgcagcaattcaaggtatgtatgtatttttattgacataattttgaagattagagatgtcatattgtttgaggtactatttgtttttgtatatatatatatatatattcactatctggtacaaaggtttgatatttcactgtctgatgaaaaggtttgatttttattttaatgttattttatgaaaaagatttttattttgaaaattagaaacgtcatatcgtttgaggggtactatatttttttttatatgttttactctttgctaagaaggtttgatctttgattattgctatataaagaaaaagagcttgatttcttgaagattagaaatgtcatatcgcttaaggggtactattttgtttttttatatgttttattgtttgttaaagaaaatttgatctttgattaatgatgtattaagaattaaaaagatcttgatttcttaaagattagaaatgtcatatcgtttgaggggtattatgtcttttgtatatatatggtatatcgtatgctaagaaggtttgatctttgaatatactatattatgaaaaagatcttgatttcttgaagagtagaaatgtaatatcgatagaggggtatctgtgatcgcatatgttgatctttaattgatgttattttatgaaaaatgattttcattttttaaagattatactctggtaataaagaagatatgatctttaaaagaattttgatttcttgaaaattagagatattatatcgatcgaagggtataacaatcgtatttgttctactgtctgctaaaaaggtttgatctaagatggatgctattttatgaaaaataaaaaatatttaaaaatttgaatcgtcactaatattatatttatatttttaatctgtgtatcgaggattgtttcttatcgtatatatgatcaaaactcaatttttttcaaagattagagaggtaagtagtactatttttctatatttagacttttgattgaaggatgttgcttatcatatttatggtaggagaaaaaaaaatgattttggactaagttttccatatactttgactttatcatatgttgaaatttttgtcaacaaagaaaatattttattggaacaaacatatgatctaggtttttaaagtagattatttcaaattaattataccattacaaatgaagaatcttgatgaatttatatatttaaagataaaagaaataattgttataatttttataaggaataatatatatgttagttgaggggagtctttaactttggtgttgatttgatgtgcaccttcatatttctccattttatgaaactttatttttcttaaagtacagacatataataaagacaagcgtgacaatttttcatataaagctgaataaattcttcatactacattataagcaaatagaatatatctagatttgatcatcatcactagtattgaaataaatcacttctttagtttactttttaacgataataatcgtgatgaaatagataaatttattcattcataacaaaaattttcaaattcgaattttgaggtgataatttatgtggaatattctaaaaaaaatcaggattaaatttatatgaatatctcaaaattatttatcttatatgaaaatggtgaattataattaccatgaccaaagataaataaaaaaggaaaaaataactaatattatctttttcaaactttcacaaaagtaatagggatcaaatcaactagtagtagtacactttatcatgcataagaaataaattgttccaaatttatacttatacaatattttgtcatggtatcttgtagtctcctacactattattatcattataaaaaagtaatacaaatttcttgattttccatccggtgctccgttttgatttttagagtatcgtttaatttaaattgtatcacgtaaaagaaaagcactttcaatatcgttataatttttttttgttttcagagattaaattcaagtcttttgattcaaggaaatgattatgagatcctttgtgttagacatgtgaatttaggaagtaaatgattattagacaacaccatgcataatttatgagcttatatttagcttttaaatataagttatttttagaaaaaacttagacaactaatttgcaacgatagaaatacaattttagagaaataaaatcatcataaaaatataggaaaattaggaaaattaaacatcaattaatgaaaaaaaaaggaagttaaatttgtgggctcgagggagtaattaaagtagattaaaagttctttttctgaattttacctgccctttgttattacttttccttttttttttttactttccatagttaatttcctttcgtttatttttttcaaaaacttttacagtaaaacaagtcaataaatatttatttcgctataaatcatttcataaagatgaaaatactagtactagtaataaggaatactaaaatgtgtggatttccggggaagttccaacgacacgccattaaaagttattttcaacgaaaatataatcaaaacatttttatttccatcggaagctcacatatttttttcgtgaatactcttctttacattttaatttgttttttttatatcagttgagcacttcgttttacaaaatgatcatctagacaactttgaaaatttttgtgtcacataagcatcaggtgtctaggagatacagtaaaaacgagttggaatacttagttgtcaaattgagatcaggttaaaatgtttacatctgcactttcaaattatattaaaacgtatagttgtcagctgatatcaaattatattaaaacgtatagttgtcagctgatatcaaatttaacgtttaattattctattacttgtgtttctagtatgtccaagacattggagagataccacagatacaattatggtacacttgaaggaacccaaacttcatcagattcacaggttttatttgtatttcttcctcaatattcaagcatgcaaattactcaaaaggttttcttagaaaacatcaaatagttcaattttttaaacctaaattagttagtaaaattaagagttaatggataagagcacatttaaattatcactttttgtgagttatcatataaaccttaactattcattgttttctttatctgtctaaactatcacttaatttgtattaaacacacctcaatgctgagttggtaaaatatttattcttattcgacatatgtgtgtaggccaactcaatattgatgtgtttttaaatataaactaagtgaaagttagaaatatgaaacttgtgaaaatgtaataatttaagcatatataaatagaaaaatagatagctgatatatgaaactcacgaaattacgacaatttatgtatttttcttttaattaactctaccattaaagctaataatattgatggctaattttgatgaaattgattttcttgttgtaaaaaacagaacaactaccaagagtatttgaagcttaaaacaagagtggaaatgttacaacagtctcaaaggtgagaaaaaaaattacttatttagaccaagattattagatgtaattttcaactccgaatgcattaagttgtcgctatttcacatggattggaccacataagatttgttgttgcctagagttactatttcaacgacttgaatgtgtgacctcctgatcacacgataacagatccagactattcattctttataatttattttgaaataactttttcatatttttaggcatttgctaggtgaggatttgggacaattgggcacaaaagacttggaacagcttgaacgtcaattggattcatcattgaggcaaattaggtcaacaaaggtactacttttattttacaaataatgacaaataatttaagataattattttttaaaattacgaagaaataatttgagacagagagagtatctaataaggtcttaattgatgaactactttttttaaaaaacttgttttcagacacaacacattcttgatcaacttgctgaacttcaacaaaaggtacattatatatagaaattaattaagatgacatttagttattttaattttctcaaattatattggaaatataatatgttttttttacttcttgtacaggaacaatctcttactgaaatgaacaaatctttgagaataaaggtaacaataataacttagactatagttttggcttaattttgatttacaaattactttgagatcgatcactcgactttatctactataacagtaaagtcaaattataatttcttgtctcactaaggtaacttcattatcatatatagttgtaaagtcacaaaattatttttgtttacttacgttacaataattgaactgactatattgaattgttatcataaataaagttcagtaatcgatcatatttttttttaacaataacttttctatggaattttgataattttagttggaagaacttggtgttacctttcaaacatcatggcattgtggtgagcaaagtgtacaatatagacatgaacagccttctcatcatgagggattttttcaacatgtaaattgcaataatacattgcctataaggtaatattgaaaatttctagcttttcatgaattttttttttaataaatttatacatataaggcacaatcaatacacaacaacaatattaacgtatcagtgtaacctcacaagtagggcctgaaaagagtggtatatgtatcagcattatctttagttaattcgtgaagatagaaaagttgtttataattgacctcgactctagtaaatcatatcaacataatacacacaaattcaatatctaatcaatcaaacgtgtagtatattatgtacacacatttttaaaaaatatcttattataaacatgcatatattttgaattaagaagcaattattacgtgctctgaaatcacttatgactttattagtctacttgtttgagactgagacacggttatatatatttgttgatttagttacggatacgataatgtacaacccgaaaatgcagcaccatcaacacatgatgctactggagttgtacctggatggatgctttga

(19) United States
(12) Patent Application Publication
(10) Pub. No.: US 2015/0135352 A1
(43) Pub. Date: May 14, 2015

(54) SOLANUM LYCOPERSICUM PLANTS HAVING NON-TRANSGENIC ALTERATIONS IN THE RIN GENE

(71) Applicant: NUNHEMS B.V., Nunhem (NL)

(72) Inventors: Hendrik Willem Vriezen, Haelen (NL); Franco Vecchio, Fidenza (IT)

(21) Appl. No.: 14/395,315

(22) PCT Filed: Mar. 11, 2013
(86) PCT No.: PCT/EP2013/054845

§ 371 Date: Oct. 17, 2014

Related U.S. Application Data
(60) Provisional application No. 61/625,412, filed on Apr. 17, 2012.

(30) Foreign Application Priority Data
Apr. 17, 2012 (EP) 12164418.1

(51) Int. Cl. A01H 5/08; C07K 14/415

(52) U.S. Cl. CPC A01H 5/08; C07K 14/415
USPC 800/260; 800/317.4; 426/615

(57) ABSTRACT

The present invention relates to cultivated plant of the species Solanum lycopersicum comprising a rin allele having one or more mutations, said mutations resulting in production of a mutant rin protein having reduced activity compared to wild type Rin protein.

### FIELD OF THE INVENTION

This invention relates to the field of plant biotechnology and plant breeding. Provided are Solarium lycopersicum plants comprising a rin allele having one or more mutations, said mutations resulting in production of a mutant rin protein having reduced activity compared to wild type Rin protein. The invention provides plants the fruits of which show slower fruit ripening and/or a longer shelf life compared to Solanum lycopersicum being homozygous for the wild type Rin allele. In addition, the invention provides tomato fruit, seeds, pollen, plant parts, and progeny of the Solanum lycopersicum plants of the invention. Food and food product of fruits of the plants of the invention is provided too.

The invention further provides an endogenous rin gene and rin protein encoded by said gene, having at least one human-induced non-transgenic mutation.

In another embodiment methods for making tomato plants comprising one or more mutant rin alleles in their genome are provided herein.

### BACKGROUND OF THE INVENTION

Breeding of Solanum lycopersicum aims at the production of commercial varieties optimally adapted to growing and storage conditions. A challenge breeders are facing is finding an improved balance between fruit firmness post-harvest and consumer desires in terms of taste texture and colour. These consumer desires relate strongly to fruit ripening. Fruit ripening is a complex developmental process responsible for the transformation of the seed-containing organ into a tissue attractive to seed dispersers and agricultural consumers. The changes associated with fruit ripening, in particular post-harvest softening, limit the shelf life of fresh tomatoes.

For tomato fruit growth and development, a number of consecutive phases can be discerned: floral development, pollination, then early fruit development takes place which is characterised by a high frequency of cell division and the fruit is rapidly increasing in size mainly due to cell expansion. At the end of the third phase the fruit reaches the mature green stage. During the fourth phase, fruit ripening takes place which is characterised by a change in colour and flavour as well as fruit firmness and texture.

The build-up of the characteristic red colour of the tomato fruit is caused by the accumulation of lycopene and carotene. In general, different colouration phases are distinguished: mature green, breaker, pink and red. At the breaker stage, the typical red pigmentation initiates. Red ripe stage or red ripe harvested fruit stage is the stage where the fruit has reached its mature colour on the major part of the fruit.

In addition to the colour changes, during fruit ripening enzymatic activity leads to degradation of the middle lamellar region of the cell walls which leads to cell loosening which is manifested as softening and loss of texture of the fruit. Softening of the fruit is often measured as external resistance to compression which can be quantified for example by a penetrometer or a texturometer (e.g. an Instron 3342 Single Column Testing System).

Modification of single genes known to be involved in ripening has not yet resulted in a fruit with normal ripening but minimal tissue softening.

The MADS-box transcription factor Ripening INhibitor (Rin) is an essential regulator of tomato (Solanum lycopersicum) fruit ripening but the exact mechanism by which it influences the expression of ripening-related genes remains unclear. The Rin gene encodes a genetic regulatory component necessary to trigger climacteric respiration and ripening-related ethylene biosynthesis in addition to requisite factors whose regulation is outside the sphere of ethylene influence. The only reported mutation at the rin locus arose spontaneous in a breeding line (R. Robinson and M. Tomes, Rep. Tomato Genet. Coop. 18, 36 (1968)). The homozygous rin mutation (rin/rin) effectively blocks the ripening process and results in green/yellow tomato fruits that do not produce elevated ethylene levels and do not ripen in response to exogenous ethylene. Tomatoes heterozygous for the rin allele remain firm and ripen over a protracted period permitting industrial-scale handling and expanded delivery and storage opportunities (Vrebalov et al, Science 296, 343-346 (2002)).

As this mutation, when homozygous, leads to an almost full stop in ripening, it can only be used commercially in heterozygous form, allowing a slower ripening to occur. However, taste development and colour of the heterozygous fruit is not optimal in the mutant. It is therefore an object of the invention to identify mutated rin alleles that cause delayed ripening and/or longer shelf life, without having these negative effects on fruit quality, colour and consumer desires. Such alleles cause tomato fruits to ripen in both heterozygous and homozygous form, due to the mutant allele encoding a mutant rin protein having reduced function (in contrast to the complete loss-of-function of the existing rin mutant).

### SUMMARY OF THE INVENTION

It is thus an object of the invention to generate and identify cultivated plants of the species Solanum lycopersicum having fruits that have delayed ripening and/or an extended post-harvest shelf life without or with acceptable negative effects on fruit quality, colour and consumer desires.

The invention thus relates to a cultivated plant of the species Solanum lycopersicum comprising a rin allele having one or more mutations, said mutations resulting in production of a mutant rin protein having reduced activity compared to wild type Rin protein, but which comprises sufficient function to result in ripening of the tomato fruits to the red stage when the mutant allele is present in heterozygous or homozygous form.

### GENERAL DEFINITIONS

The term “nucleic acid sequence” (or nucleic acid molecule) refers to a DNA or RNA molecule in single or double stranded form, particularly a DNA encoding a protein or protein fragment according to the invention. An “isolated nucleic acid sequence” refers to a nucleic acid sequence which is no longer in the natural environment from which it was isolated, e.g. the nucleic acid sequence in a bacterial host cell or in the plant nuclear or plastid genome.

The terms “protein” or “polypeptide” are used interchangeably and refer to molecules consisting of a chain of amino acids, without reference to a specific mode of action, size, 3-dimensional structure or origin. A “fragment” or “portion” of Rin protein may thus still be referred to as a “protein”. An “isolated protein” is used to refer to a protein which is no longer in its natural environment, for example in vitro or in a recombinant bacterial or plant host cell.

The term “gene” means a DNA sequence comprising a region (transcribed region), which is transcribed into an RNA molecule (e.g. an mRNA or an RNAi molecule) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites. A gene may be an endogenous gene (in the species of origin) or a chimeric gene (e.g. a transgene or cisgene).

“Expression of a gene” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi). The coding sequence may be in sense-orientation and encodes a desired, biologically active protein or peptide, or an active peptide fragment.

An “active protein” or “functional protein” is a protein which has protein activity as measurable in vitro, e.g. by an in vitro activity assay, and/or in vivo, e.g. by the phenotype conferred by the protein. A “wild type” protein is a fully functional protein, as present in the wild type plant. A “mutant protein” is herein a protein comprising one or more mutations in the nucleic acid sequence encoding the protein, whereby the mutation results in (the mutant nucleic acid molecule encoding) a “reduced-function” or “loss-of-function” protein, as e.g. measurable in vivo, e.g. by the phenotype conferred by the mutant allele.

A “reduced function rin protein” or “reduced activity rin protein” refers to a mutant rin protein which is still capable of causing fruit ripening to occur to the red stage when the allele encoding the mutant protein is present in homozygous form in the tomato plant. Such a reduced function rin protein can be obtained by the translation of a “partial knockout mutant rin allele” which is, for example, a wild-type Rin allele, which comprises one or more mutations in its nucleic acid sequence. In one aspect, such a partial knockout mutant rin allele is a wild-type Rin allele, which comprises a mutation that preferably result in the production of an rin protein wherein at least one conserved and/or functional amino acid is substituted for another amino acid, such that the biological activity is significantly reduced but not completely abolished. Such partial knockout mutant rin allele may also encode a dominant negative rin protein, which is capable of adversely affecting the biological activity of other Rin proteins within the same cell. Such a dominant negative rin protein can be a rin protein that is still capable of interacting with the same elements as the wild-type Rin protein, but that blocks some aspect of its function. Examples of dominant negative rin proteins are rin proteins that lack, or have modifications in, the activation domain and/or dimerization domain or specific amino acid residues critical for activation and/or dimerization, but still contain the DNA binding domain, such that not only their own biological activity is reduced or abolished, but that they further reduce the total rin activity in the cell by competing with wildtype and/or partial knockout rin proteins present in the cell for DNA binding sites. Mutant alleles can be either “natural mutant” alleles, which are mutant alleles found in nature (e.g. produced spontaneously without human application of mutagens) or “induced mutant” alleles, which are induced by human intervention, e.g. by mutagenesis.

A “loss-of-function rin protein” refers to a mutant rin protein which is not capable of causing fruit ripening to occur to the red stage when the allele encoding the mutant rin protein is present in homozygous form in the tomato plant, such as the existing rin mutation present in the prior art (described e.g. by Vrebalov et al. 2002, Science 296: 343-346; Ito et al., 2008, Plant J. 55: 212-223; Martel et al. 2011, Plant Physiol 157: 1568-1579; and also Accession LA3754 has the prior art rin/rin, obtainable from TGRC, Tomato Genetics Resource Centre).

A “mutation” in a nucleic acid molecule coding for a protein is a change of one or more nucleotides compared to the wild type sequence, e.g. by replacement, deletion or insertion of one or more nucleotides. A “point mutation” is the replacement of a single nucleotide, or the insertion or deletion of a single nucleotide.

A “nonsense” mutation is a (point) mutation in a nucleic acid sequence encoding a protein, whereby a codon is changed into a stop codon. This results in a premature stop codon being present in the mRNA and in a truncated protein. A truncated protein may have reduced function or loss of function.

A “missense” or non-synonymous mutation is a (point) mutation in a nucleic acid sequence encoding a protein, whereby a codon is changed to code for a different amino acid. The resulting protein may have reduced function or loss of function.

A “splice-site” mutation is a mutation in a nucleic acid sequence encoding a protein, whereby RNA splicing of the pre-mRNA is changed, resulting in an mRNA having a different nucleotide sequence and a protein having a different amino acid sequence than the wild type. The resulting protein may have reduced function or loss of function.

A “frame-shift” mutation is a mutation in a nucleic acid sequence encoding a protein by which the reading frame of the mRNA is changed, resulting in a different amino acid sequence. The resulting protein may have reduced function or loss of function.

A mutation in a regulatory sequence, e.g. in a promoter of a gene, is a change of one or more nucleotides compared to the wild type sequence, e.g. by replacement, deletion or insertion of one or more nucleotides, leading for example to reduced or no mRNA transcript of the gene being made.

“Silencing” refers to a down-regulation or complete inhibition of gene expression of the target gene or gene family.

A “target gene” in gene silencing approaches is the gene or gene family (or one or more specific alleles of the gene) of which the endogenous gene expression is down-regulated or completely inhibited (silenced) when a chimeric silencing gene (or ‘chimeric RNAi gene’) is expressed and for example produces a silencing RNA transcript (e.g. a dsRNA or hairpin RNA capable of silencing the endogenous target gene expression). In mutagenesis approaches, a target gene is the endogenous gene which is to be mutated, leading to a change in (reduction or loss of) gene expression or a change in (reduction or loss of) function of the encoded protein.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous and, where necessary to join two protein encoding regions, contiguous and in reading frame so as to produce a “chimeric protein”. A “chimeric protein” or “hybrid protein” is a protein composed of various protein “domains” (or motifs) which is not found as such in nature but which a joined to form a functional protein, which displays the functionality of the joined domains. A chimeric protein may also be a fusion protein of two or more proteins occurring in nature.

The term “food” is any substance consumed to provide nutritional support for the body. It is usually of plant or animal origin, and contains essential nutrients, such as carbohydrates, fats, proteins, vitamins, or minerals. The substance is ingested by an organism and assimilated by the organism's cells in an effort to produce energy, maintain life, or stimulate growth. The term food includes both substance consumed to provide nutritional support for the human and animal body.

The term “shelf life” “post-harvest shelf life” designates the (average) length of time that a fruit is given before it is considered unsuitable for sale or consumption (‘bad’). Shelf life is the period of time that products can be stored, during which the defined quality of a specified proportion of the goods remains acceptable under expected conditions of distribution, storage and display. Shelf life is influenced by several factors: exposure to light and heat, transmission of gases (including humidity), mechanical stresses, and contamination by e.g. micro-organisms. Product quality is often mathematically modelled around the fruit firmness/softness parameter. Shelf-life can be defined as the (average) time it takes for fruits of a plant line to start to become had and unsuitable for sale or consumption, starting for example from the first fruit of a plant entering breaker stage or turning stage or from the first fruit becoming fully red or from harvest. In one embodiment the mutants according to the invention have a shelf life that is significantly longer than the shelf life of wild type plants, for example the number of days from the first fruit being in breaker stage (or turning stage, pink stage, red stage or from harvest) up to the first fruit starting to become ‘bad’ and unsuitable for sale or consumption is significantly longer, e.g. at least 1, 2, 3, 4, 5, 6, 7, 8, 9, 10, or more, days longer than fruits of control plants (such as wild type Rin/Rin plants), when plants are grown under the same conditions and fruits are treated the same way and kept under the same conditions. Thus, to determine the number of days required from a certain stage (e.g. from breaker stage or a later stage) to ‘bad’ stage, the day when the first fruit of the wild type control plant (grown under the same conditions as the mutant plants and being at the same developmental stage) enters a certain stage (e.g. breaker stage or a later stage) can, for example, be taken as the starting point (day 1) from when on periodically (at certain time intervals, e.g. after 1, 2, 3, 4, 5 or 6 days) the fruits are observed until the day that the first fruit has passed the fully ripe stage and becomes ‘bad’ (as determinable visually and/or through assessing fruit softness) (see Examples).

In this application the words “improved”, “increased”, “longer” and “extended” as used in conjunction with the word “shelf-life” are interchangeable and all mean that the fruits of a tomato plant according to the invention have on average, a longer shelf-life than the control fruits (Rin/Rin fruits).

“Delayed ripening” means that the fruits of a tomato plant or plant line (e.g. a mutant) according to the invention require on average significantly more days to reach the red stage from the mature green, breaker, turning stage, and/or pink stages of tomato fruit ripening compared to wild type control fruits of plants homozygous for the wild type Rin allele (Rin/Rin). Delayed ripening can be measure on the plant and/or after harvest as days required for a certain percentage of fruits (e.g. 10%, 20%, 30% 40%, 50%, 60%, 70%, 80%, 90% and/or 100% of fruits) to reach the red stage. A plant is said to have a delayed ripening phenotype if it takes at least 2, 3, 4, 5, 6, 7, 8, 9, 10, 11, 12, 13, 14 or 15 days longer for 10%, 20%, 30% 40%, 50%, 60%, 70%, 80%, 90% and/or 100% of fruits to reach the red stage than it takes for the wild type control fruits to develop the same percentage of red fruits. The day when the first fruit of the wild type control plant (grown under the same conditions as the mutant plants and being at the same developmental stage) enters a certain stage (e.g. breaker stage) can, for example, be taken as the starting point (day 1) from when on periodically (at certain time intervals (e.g. after 1, 2, 3, 4, 5 or 6 days) the number of fruits that are in breaker stage and the number of fruit that are in red stage are counted, both for the mutant plant line and control plants (see Examples).

It is understood that comparisons between different plant lines involves growing a number of plants of a line (e.g. at least 5 plants, preferably at least 10 plants per line) under the same conditions as the plants of one or more control plant lines (preferably wild type Rin/Rin plants) and the determination of statistically significant differences between the plant lines when grown under the same environmental conditions.

“Delay of breaker stage” refers to the mutants according to the invention requiring significantly more days than wild type Rin/Rin controls for the first fruits and/or for all fruits to have entered breaker stage, e.g. at least 1 more day, preferably at least 2, 3, 4, 5, 6, 7, 8, 9, 10, 11 or 12 more days than the wild type control, when grown under the same conditions.

The “ripening stage” of a tomato fruit can be divided as follows: (1) Mature green stage: surface is completely green; the shade of green may vary from light to dark. (2) Breaker stage: there is a definite break in color from green to tannish-yellow, pink or red on not more than 10% of the surface; (3) Turning stage: 10% to 30% of the surface is not green; in the aggregate, shows a definite change from green to tannish-yellow, pink, red, or a combination thereof. (4) Pink stage: 30% to 60% of the surface is not green; in the aggregate, shows pink or red color. (5) Light red stage: 60% to 90% of the surface is not green; in the aggregate, shows pinkish-red or red. (6) Red stage: More than 90% of the surface is not green; in the aggregate, shows red color.

“Sequence identity” and “sequence similarity” can be determined by alignment of two peptide or two nucleotide sequences using global or local alignment algorithms. Sequences may then be referred to as “substantially identical” or “essentially similar” when they are optimally aligned by for example the programs GAP or BESTFIT or the Emboss program “Needle” (using default parameters, see below) share at least a certain minimal percentage of sequence identity (as defined further below). These programs use the Needleman and Wunsch global alignment algorithm to align two sequences over their entire length, maximizing the number of matches and minimises the number of gaps. Generally, the default parameters are used, with a gap creation penalty=10 and gap extension penalty=0.5 (both for nucleotide and protein alignments). For nucleotides the default scoring matrix used is DNAFULL and for proteins the default scoring matrix is Blosum62 (Henikoff & Henikoff, 1992, PNAS 89, 915-919). Sequence alignments and scores for percentage sequence identity may for example be determined using computer programs, such as EMBOSS (http://www.ebi.ac.uk/Tools/psa/emboss_needle/). Alternatively percent similarity or identity may be determined by searching against databases such as FASTA, BLAST, etc., but hits should be retrieved and aligned pairwise to compare sequence identity. Two proteins or two protein domains, or two nucleic acid sequences have “substantial sequence identity” if the percentage sequence identity is at least 90%, 95%, 98%, 99% or more (as determined by Emboss “needle” using default parameters, i.e. gap creation penalty=10, gap extension penalty=0.5, using scoring matrix DNAFULL for nucleic acids an Blosum62 for proteins). Such sequences are also referred to as ‘variants’ herein, e.g. other variants of mutant rin alleles and mutant rin proteins than the specific nucleic acid and protein sequences disclosed herein can be identified, which have the same effect on delayed ripening and/or longer shelf-life of the fruits comprising such variants.

The “MADS-box” or “MADS-domain” or “MADS-box domain” and “K-box” or “K-domain” or K-box domain refers to the protein domain as determinable by entering an amino acid sequence in the PROSITE pattern scan on e.g. http://prosite.expasy.org/. For SEQ ID NO: 1 (wild type Rin protein), the MADS-box comprises amino acid 1 to 61 and the K-domain comprises amino acids 87 to 177. Functional MADS-box domains or functional K-box domains may also exist in other normally ripening tomato plants comprising functional variants of SEQ ID NO: 1, which comprise e.g. 1, 2 or 3 amino acid insertions, deletions or replacements but do not reduce functionality of the Rin protein (and are thus considered to be wild type, functional Rin proteins and functional MADS-box or K-box domains).

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. It is further understood that, when referring to “sequences” herein, generally the actual physical molecules with a certain sequence of subunits (e.g. amino acids) are referred to.

As used herein, the term “plant” includes the whole plant or any parts or derivatives thereof, such as plant organs (e.g., harvested or non-harvested fruits, flowers, leaves, etc.), plant cells, plant protoplasts, plant cell or tissue cultures from which whole plants can be regenerated, regenerable or non-regenerable plant cells, plant calli, plant cell clumps, and plant cells that are intact in plants, or parts of plants, such as embryos, pollen, ovules, ovaries, fruits (e.g., harvested tissues or organs, such as harvested tomatoes or parts thereof), flowers, leaves, seeds, tubers, clonally propagated plants, roots, stems, cotyledons, hypocotyls, root tips and the like. Also any developmental stage is included, such as seedlings, immature and mature, etc.

A “plant line” or “breeding line” refers to a plant and its progeny. As used herein, the term “inbred line” refers to a plant line which has been repeatedly selfed.

“Plant variety” is a group of plants within the same botanical taxon of the lowest grade known, which (irrespective of whether the conditions for the recognition of plant breeder's rights are fulfilled or not) can be defined on the basis of the expression of characteristics that result from a certain genotype or a combination of genotypes, can be distinguished from any other group of plants by the expression of at least one of those characteristics, and can be regarded as an entity, because it can be multiplied without any change. Therefore, the term “plant variety” cannot be used to denote a group of plants, even if they are of the same kind, if they are all characterized by the presence of 1 locus or gene (or a series of phenotypical characteristics due to this single locus or gene), but which can otherwise differ from one another enormously as regards the other loci or genes.

“F1, F2, etc.” refers to the consecutive related generations following a cross between two parent plants or parent lines. The plants grown from the seeds produced by crossing two plants or lines is called the F1 generation. Selfing the F1 plants results in the F2 generation, etc. “F1 hybrid” plant (or F1 seed) is the generation obtained from crossing two inbred parent lines. An “M1 population” is a plurality of mutagenized seeds/plants of a certain plant line or cultivar. “M2, M3, M4, etc.” refers to the consecutive generations obtained following selfing of a first mutagenized seed/plant (M1).

The term “allele(s)” means any of one or more alternative forms of a gene at a particular locus, all of which alleles relate to one trait or characteristic at a specific locus. In a diploid cell of an organism, alleles of a given gene are located at a specific location, or locus (loci plural) on a chromosome. One allele is present on each chromosome of the pair of homologous chromosomes. A diploid plant species may comprise a large number of different alleles at a particular locus. These may be identical alleles of the gene (homozygous) or two different alleles (heterozygous).

The term “locus” (loci plural) means a specific place or places or a site on a chromosome where for example a gene or genetic marker is found. The RIN locus is thus the location in the genome where the RIN gene is found.

“Wild type allele” (WT) refers herein to a version of a gene encoding a fully functional protein (wild type protein). Such a sequence encoding a fully functional Rin protein is for example the wild type Rin cDNA (mRNA) sequence depicted in SEQ ID NO: 5, based on Genbank AF448522. The protein sequence encoded by this wild type Rin mRNA is depicted in SEQ ID NO: 1. It consists of 242 amino acids. Two domains have been mentioned to occur on the Rin protein i.e. a MADS domain, presumed to be involved in DNA binding (amino acid 1-61), and the K-box domain, presumed to be involved in protein-protein interaction (amino acid 87-177 of SEQ ID NO: 1, corresponding to the last two amino acids of exon 2 up to the first 7 amino acids of exon 7). Other fully functional Rin protein encoding alleles (i.e. alleles which confer ripening to the same extent as the protein of SEQ ID NO 1) may exist in other Solanum lycopersicum plants and may comprise substantial sequence identity with SEQ ID NO: 1, i.e. at least about 90%, 95%, 98% or 99% sequence identity with SEQ ID NO: 1. Such fully functional wild type Rin proteins are herein referred to as variants of SEQ ID NO: 1. Likewise the nucleotide sequences encoding such fully functional Rin proteins are referred to as variants of SEQ ID NO: 5 or of SEQ ID NO: 9.

The genomic Rin DNA is depicted in SEQ ID NO: 9. It contains 8 exons interrupted by 7 introns. Exons 1-8 are located from nucleotides 1-185, 3060-3138, 3653-3714, 3941-4040, 4182-4223, 4323-4364, 4654-4787, and 5202-5286 of SEQ ID NO:9, respectively.

The following mutant rin alleles are exemplary of the delayed-ripening and/or extended shelf-life conferring rin mutations identified according to the present invention. One exemplary mutant rin allele comprises a T to C mutation at nucleotide 3949 of SEQ ID NO: 9 (mutant 5996), counting A in the ATG of the START CODON as nucleotide position 1. This causes a T to C at nucleotide 335 of the wild type cDNA sequence SEQ ID NO: 5, again counting A in the ATG of the START CODON as nucleotide position 1, which is within exon 4 of the rin gene. This mutation results in a change from leucine to proline at amino acid 112 in the encoded protein (SEQ ID NO: 4). The Leu112Pro mutation is within the K-domain of the Rin protein. The protein sequence of mutant 5996 is depicted in SEQ ID NO: 4. The corresponding cDNA is depicted in SEQ ID NO: 8.

Another exemplary mutant rin allele conferring delayed ripening and/or extended shelf-life identified according to the present invention, comprises with a G to A mutation at nucleotide 3692 of SEQ ID NO: 9 (mutant 5225), counting A in the ATG of the START CODON as nucleotide position 1.

This causes a G to A replacement at nucleotide 304 of SEQ ID NO: 5, again counting A in the ATG of the START CODON as nucleotide position 1. This mutation results in a change from glutamic acid to lysine at amino acid 102 in the encoded protein (SEQ ID NO: 3). The Glu102Lys mutation is within the K-domain of the Rin protein. The protein sequence of mutant 5225 is depicted in SEQ ID NO: 3. The corresponding mutant cDNA is depicted in SEQ ID NO: 7.

Still another exemplary mutant rin allele conferring delayed ripening and/or extended shelf-life, identified according to the present invention, comprises a change of G to A at nucleotide 3652 of SEQ ID NO: 9 (mutant 2558) counting A in the ATG of the START CODON as nucleotide position 1. Mutant 2558 carries a mutation in the last nucleotide before the splicing acceptor site between intron 2 and exon 3. Such a mutation close to a splice site may cause miss-splicing. In the present case, the mutation is just before the beginning of exon 3 and the corresponding cDNA (SEQ ID NO: 6) lacks 62 nucleotides (corresponding to exon 3). This causes a frame-shift in the reading of exon 4, which leads to a stop codon (TGA) after the 4^{th} codon in exon 4. The truncated protein is depicted in SEQ ID NO: 2 and comprises the amino acids encoded by exons 1 and 2. It still contains the complete MADS-domain but lost the entire K-box domain and the C-terminus of the protein.

“Mutant allele” refers herein to an allele comprising one or more mutations in the coding sequence (mRNA, cDNA or genomic sequence) compared to the wild type allele. Such mutation(s) (e.g. insertion, inversion, deletion and/or replacement of one or more nucleotides) may lead to the encoded protein having reduced in vitro and/or in vivo functionality (reduced function) or no in vitro and/or in vivo functionality (loss-of-function), e.g. due to the protein e.g. being truncated or having an amino acid sequence wherein one or more amino acids are deleted, inserted or replaced. Such changes may lead to the protein having a different 3D conformation, being targeted to a different sub-cellular compartment, having a modified catalytic domain, having a modified binding activity to nucleic acids or proteins, etc.

“Wild type plant” and “wild type fruits” or “normal ripening” plants/fruits refers herein to a tomato plant comprising two copies of a wild type (WT) Rin allele (Rin/Rin) encoding a fully functional Rin protein (e.g. in contrast to “mutant plants”, comprising a mutant rin allele). Such plants are for example suitable controls in phenotypic assays. Preferably wild type and/or mutant plants are “cultivated tomato plants”. For example the cultivar Moneymaker is a wild type plant, cultivar Ailsa Craig, as is inbred line TPAADASU (Gady et al. 2009, Plant Methods 5:13 and Gady et al. 2012, Mol Breeding 29(3): 801-812) and many others. Plants homozygous for wild type Rin can also be obtained from selfing commercial hybrids (e.g. Daniella, Red Centre, Nada F1, Sampion F1, Carmen F1, Chronos F1) which are heterozygous, Rin/rin, and selecting the Rin/Rin progeny.

“Tomato plants” or “cultivated tomato plants” are plants of the Solanum lycopersicum, i.e. varieties, breeding lines or cultivars of the species Solanum lycopersicum, cultivated by humans and having good agronomic characteristics; preferably such plants are not “wild plants”, i.e. plants which generally have much poorer yields and poorer agronomic characteristics than cultivated plants and e.g. grow naturally in wild populations. “Wild plants” include for example ecotypes, PI (Plant Introduction) lines, landraces or wild accessions or wild relatives of a species. The so-called heirloom varieties or cultivars, i.e. open pollinated varieties or cultivars commonly grown during earlier periods in human history and often adapted to specific geographic regions, are in one aspect of the invention encompassed herein as cultivated tomato plants.

Wild relatives of tomato include S. arcanum, S. chmielewskii, S. neorickii (=L. parviflorum), S. cheesmaniae, S. galapagense, S. pimpinellifolium, S. chilense, S. corneliomulleri, S. habrochaites (=L. hirsutum), S. huaylasense, S. sisymbriifolium, S. peruvianum, S. hirsutum or S. pennellii.

“Average” refers herein to the arithmetic mean.

### BRIEF DESCRIPTION OF THE SEQUENCE LISTING

SEQ ID NO: 1 shows the Solanum lycopersicum wild type Rin protein sequence as derived from the mRNA based on Genbank Accession number AF448522.

SEQ ID NO: 2 shows the Solanum lycopersicum mutant 2558 rin protein.

SEQ ID NO: 3 shows the Solanum lycopersicum mutant 5225 rin protein.

SEQ ID NO: 4 shows the Solanum lycopersicum mutant 5996 rin protein.

SEQ ID NO: 5 shows the Solanum lycopersicum wild type Rin cDNA (Genbank Accession AF448522).

SEQ ID NO: 6 shows the Solanum lycopersicum mutant 2558 rin cDNA.

SEQ ID NO: 7 shows the Solanum lycopersicum mutant 5225 rin cDNA.

SEQ ID NO: 8 shows the Solanum lycopersicum mutant 5996 rin cDNA.

SEQ ID NO: 9 shows the Solanum lycopersicum genomic Rin DNA, and the wild type Rin protein.

### BRIEF DESCRIPTION OF THE FIGURES

FIG. 1: In this graph the percentage of fruits in red stage is shown, determined at various days after the wild type control fruits started entering breaker stage. All fruits of mutant plants of the invention require more days to ripen compared to wild type (wt), homozygous for the wild type Rin allele (Rin/Rin). ‘Ho’ means fruits of a mutant plant (indicated by the preceding number) being homozygous for a specific rin mutation (rin/rin); He means fruits of a mutant (indicated by the preceding number) being heterozygous for a specific rin mutation (Rin/rin).

FIG. 2: Ethylene-release measured in n1/(h·g), also written as n1·h⁻¹·g⁻¹, from tomato fruits at Pink stage and Red stage (wherein ‘g’ refers to grams fresh weight). Tapa is the wild type control, a highly homozygous inbred parental line used in commercial processing tomato breeding (Gady et al. 2009, Plant Methods 5:13 and Gady et al. 2012, Mol Breeding 29(3): 801-812) and is homozygous for the wild type rin allele (Rin/Rin). Mutants 2558 and 5996 are both homozygous for mutated rin allele.

FIG. 3A-H: NRQ values for various primer combinations (as explained in Example 4) for Wild Type (WT), existing rin mutant (rin), plants according to the invention 2558, 5225, 5996 at Mature Green (MG) and Breaker Stage (BR) stage.

### DETAILED DESCRIPTION OF THE INVENTION

The present invention discloses a cultivated plant of the species Solanum lycopersicum comprising a rin allele having one or more mutations, said mutations resulting in production of a mutant rin protein having reduced function compared to wild type Rin protein.

The Solanum lycopersicum (tomato) wild type Rin gene comprises 8 exons separated by 7 introns (see SEQ ID NO: 9) and 5′ and 3′ untranslated regions.

The Rin protein sequence contains 2 domains: a MADS domain and a K-box domain. The MADS box domain is presumed necessary for DNA binding and protein interactions and ranges from amino acid 1-61 of SEQ ID NO: 1. The K-box domain is important to strengthen the activity of the MADS domain and is presumed to be involved in protein-protein interaction. It covers amino acids 87-177 of SEQ ID NO: 1.

In one aspect the invention relates to a cultivated plant of the species Solanum lycopersicum, and parts thereof (e.g. fruits), comprising a rin allele having one or more mutations, said mutations resulting in production of a mutant rin protein having reduced function compared to wild type Rin protein wherein said mutation or mutations result in delayed fruit ripening and/or a longer shelf life compared to Solanum lycopersicum plants which are homozygous for the wild type fully functional Rin allele (Rin/Rin) (encoding a functional Rin protein of SEQ ID NO: 1 or a functional variant).

In another aspect, the mutation or mutations in the plant of the invention result in delayed fruit ripening and/or a longer shelf life compared to Solanum lycopersicum being homozygous for the wild type Rin allele.

In yet another aspect, the invention relates to a cultivated plant of the species Solanum lycopersicum comprising a rin allele having one or more mutations resulting in a reduced-function rin protein, wherein said mutation(s) are not occurring in the MADS domain, i.e. no mutation in the first 61 amino acid-encoding part of the wild type, functional Rin protein encoding, Rin allele, and said mutations resulting in production of a mutant rin protein having reduced function compared to wild type Rin protein wherein said mutation or mutations result in delayed fruit ripening and/or a longer shelf life compared to Solanum lycopersicum being homozygous for the wild type Rin allele.

The Solanum lycopersicum plant thus comprises a rin allele encoding a reduced-function rin protein, which protein comprises a functional MADS-domain, i.e. the mutation leading to the delayed ripening and/or longer shelf life, lies outside the MADS-domain. Thus, in one embodiment the mutant rin allele encodes the N-terminus of SEQ ID NO: 1 from amino acid 1 to 61, or the N-terminus of a variant of SEQ ID NO:1 from amino acid 1 to 61 which comprises a functional MADS-domain, and further comprises (a nucleotide sequence encoding) at least one amino acid insertion, deletion or replacement in amino acids 62 to 242 of SEQ ID NO: 1, said at least one insertion, deletion or replacement leading to a delay in ripening and/or longer shelf life of the fruit of the tomato plant. Yet, the fruits do ripen to red stage, i.e. the amino acid insertion, deletion or replacement does not lead to an abolishment of ripening when the allele is present in homozygous form. The reduced function rin protein according to the invention are not loss-of-function rin proteins, as is described for the existing rin/rin mutant plants which fail to ripen and remain green or yellowish.

In one embodiment the mutation(s) causing the reduced-function of the rin protein is/are in the K-domain of the wild type Rin protein, thus in one embodiment one or more amino acids are inserted, deleted or replaced in amino acids 87 to 177 of SEQ ID NO: 1 (or a variant of SEQ ID NO: 1). In another embodiment the mutation(s) causing the reduced-function of the rin protein is/are in the C-terminus of the wild type Rin protein, thus in one embodiment one or more amino acids are inserted, deleted or replaced in amino acids 178 to 242 of SEQ ID NO: 1 (or a variant of SEQ ID NO: 1).

The existing prior art rin/rin mutation is due to a deletion of 1.7 kb ranging from part of intron 7 and the complete exon 8 through to the nearby gene MC. As a result a fusion protein is produced which comprises exons 1-7 of Rin fused to the MC protein. This fusion protein is not functional in vivo, i.e. the fruits do not ripen in rin/rin plants and also no transcriptional activation of genes which (functional, wild type) RIN protein activates takes place. This mutation is a loss-of-function mutant.

Thus, in one embodiment of the invention, the tomato plants according to the invention comprise an endogenous (non-transgenic) mutant rin allele, which encodes a reduced-function mutant rin protein (not a loss-of-function mutant), whereby the fruits of the plant do ripen to the red stage (albeit slower than plants homozygous for the wild type, fully functional Rin protein) and whereby also transcriptional activation of Rin-induced genes takes place in the fruits, either homozygous or heterozygous for the mutant Rin protein. To measure transcriptional activation of Rin-induced genes the mRNA levels or the relative gene expression levels of the following genes can be measured at various ripening stages (especially at breaker stage and thereafter), using e.g. quantitative RT-PCR: ACS2, ACS4, NR, E8, E4 (all ethylene synthesis, perception and response genes) and PSY1 (carotenoid biosynthesis gene). See Martel et al. (2011, supra). Thus, at least these genes are expressed in the heterozygous or homozygous mutant fruits according to the invention, while they are not expressed in a homozygous loss-of-function rin mutant fruits.

In still another aspect, the invention relates to a plant according to the invention having an endogenous rin allele encoding a reduced-function rin protein having substantial sequence identity to SEQ. ID NO: 1, or to a variant of SEQ ID NO: 1, wherein said protein comprising one or more amino acid replacements, deletions and/or insertions.

In yet another aspect, the invention relates to a plant of the invention comprising delayed ripening and/or longer shelf-life than wild type (Rin/Rin) plants, due to said plants comprising an endogenous rin allele encoding a reduced-function rin protein having substantial sequence identity to SEQ. ID NO: 2 or to SEQ. ID NO: 3, or to SEQ. ID NO: 4. In a specific aspect, the invention relates to cultivated tomato plants comprising a rin allele as found in seed deposited under accession number NCIMB 41937, NCIMB 41938 or NCIMB 41939 in one or two copies, i.e. in homozygous or heterozygous form. In heterozygous form, the other allele may be a wild type Rin allele or another mutant rin allele, such as from any one of the other mutants provided herein, or any other mutant rin allele encoding for a reduced-function rin protein as described herein. The other allele is preferably not a loss-of-function rin allele.

In yet another aspect, the invention relates to a tomato plant of the invention comprising an endogenous rin allele encoding a reduced-function rin protein having 100% sequence identity to SEQ. ID NO: 2, or to SEQ. ID NO: 3, or to SEQ. ID NO: 4.

In yet a further aspect, the invention relates to a plant of the invention comprising an endogenous rin allele encoding a reduced-function rin protein having at least one amino acid deletion, insertion or replacement in the K-box domain. Preferably the rin protein comprises a functional MADS-domain, such as the MADS domain of SEQ ID NO: 1 (amino acids 1-61) or the MADS-domain of a (functional) variant of SEQ ID NO: 1. In one embodiment it also comprises the C-terminal of SEQ ID NO: 1 (amino acids 178-242) or the C-terminal of a (functional) variant of SEQ ID NO: 1. In one aspect, the rin protein is not longer than 242 amino acids. It does not further comprise a fusion with all or part of another protein attached to the rin protein. The functional MADS-domain may thus be the MADS-domain of SEQ ID NO: 1 or a MADS-domain with substantial sequence identity to the MADS-domain of SEQ ID NO: 1. The invention further relates to tomato seeds, plants and plant parts comprising an endogenous rin gene having substantial sequence identity to SEQ. ID NO: 9 and having at least one non-transgenic mutation within said endogenous rin gene, wherein said at least one non-transgenic mutation results in the production of a mutant rin protein having reduced activity compared to wild type Rin protein. Preferably, said mutation results in slower fruit ripening and/or a longer shelf life compared to Solanum lycopersicum being homozygous for the wild type Rin allele. The mutation described anywhere herein may be human-induced or it may be a natural mutation. The plant is preferably a cultivated tomato plant. In another embodiment, said mutation is selected from the group consisting of T3949C, G3692A and G2652A of SEQ ID NO: 9.

In another aspect the invention relates to tomato seeds, plants and plant parts comprising an endogenous mutant rin gene wherein said non-transgenic mutation creates an amino acid change in the rin protein encoded by and produced by transcription and translation of the rin gene, wherein said amino acid change is selected from the group consisting of Leu112Pro, Glu102Lys and the complete deletion of exon 3 (amino acids 89 to 109 of SEQ ID NO: 1). Such a deletion of exon 3 may be caused by a splice site mutation. Said splice site mutation may be in intron 2, e.g. just before the start of exon 3. The splice-site mutation may be a mutation in the last 1, 2, 3, 4, 5, or 6 nucleotides before exon 3 (nucleotides 3647 to 3652 of SEQ ID NO: 9).

In yet another aspect the invention relates to rin protein having substantial sequence identity to SEQ ID NO: 2. In still another aspect the invention relates to rin protein having substantial sequence identity to SEQ ID NO: 3. In a further aspect the invention relates to rin protein having substantial sequence identity to SEQ ID NO: 4. The invention also relates to tomato seeds, plants and plant parts comprising a nucleotide sequence encoding these proteins.

In still another aspect, the invention relates to tomato fruit, seeds, pollen, plant parts, and/or progeny of a plant of the invention. Preferably, the invention relates to fruit or seeds of the plant of the invention. More preferably, the invention relates to tomato fruit having delayed ripening and/or an increased post-harvest shelf life caused by a non-transgenic mutation in at least one rin allele, as described elsewhere herein

In one aspect the tomato plants according to the invention have a delay of breaker stage, meaning that the mutants according to the invention requiring significantly more days than wild type Rin/Rin controls for the first fruits and/or for all fruits to have entered breaker stage.

In a particular aspect the tomato plants according to the invention have a shelf life that is significantly longer than the shelf life of wild type plants, for example the number of days from the first fruit being in breaker stage (or turning stage, pink stage, red stage or from harvest) up to the first fruit starting to become ‘bad’ and unsuitable for sale or consumption is significantly longer, e.g. at least 1, 2, 3, 4, 5, 6, 7, 8, 9, 10, or more, days longer than fruits of control plants (such as wild type Rin/Rin plants), when plants are grown under the same conditions and fruits are treated the same way and kept under the same conditions.

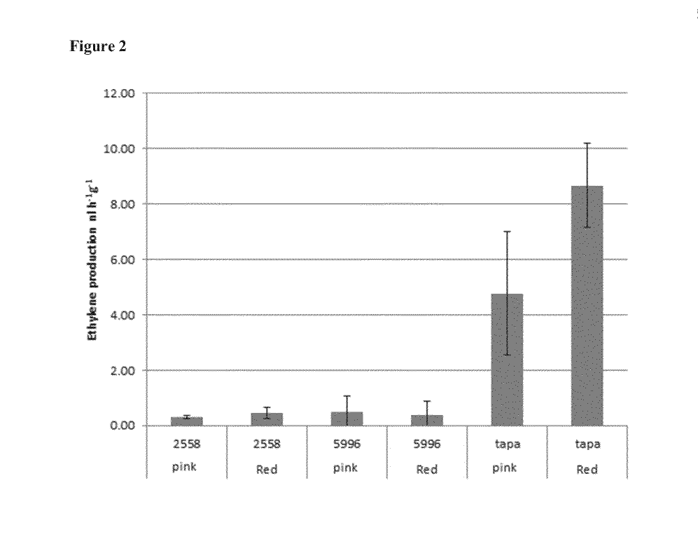

A delayed ripening and/or extended shelf-life can have the advantage that more time is available for transport of picked fruits e.g. to retailers and supermarkets and/or that the consumer can keep the fruits longer. Tomatoes can be harvested at mature green stage or at breaker stage, or thereafter. When harvested before breaker stage, ethylene exposure is needed, while harvest around breaker stage or thereafter does not require ethylene exposure, as the fruits produce ethylene themselves. As seen in FIG. 2, delayed-ripening mutants according to the invention produce less ethylene at pink stage and red stage than wild type fruits, but sufficient ethylene to ripen to the red stage. In one aspect of the invention tomato plants are provided comprising a mutant rin allele encoding a reduced function rin protein, wherein the fruits of said plants produce significantly less ethylene than wild type (Rin/Rin) plants (but significantly more ethylene than loss-of-function rin/rin mutants). “Significantly less ethylene” refers to the fruit producing equal to or less than 50%, equal to or less than 40%, equal to or less than 30%, equal to or less than 20% of the ethylene produced by homozygeous Rin/Rin fruits at the pink or red stage. Thus, the ethylene produced at the pink stage or at the red stage is in one aspect below about 2 n1/(h·g), such as equal to or below about 1 n1/(h·g) or equal to or below about 0.5 n1/(h·g).

In another aspect, the invention relates to tomato fruit of a plant of the invention having a longer ripening period and/or an increased post-harvest shelf life caused by a non-transgenic mutation in at least one rin allele wherein the longer ripening period and/or the longer post-harvest shelf life is at least 110% of the ripening period and/or of the post-harvest shelf life of a tomato fruit being homozygous for the wild type Rin allele. Preferably, the ripening period and/or post-harvest shelf life is at least 115%, more preferably at least 120%, even more preferably at least 125% of the ripening period and/or post-harvest shelf life of a tomato fruit being homozygous for the wild type Rin allele. In another aspect, the ripening period and/or post-harvest shelf life is at least 135%, more preferably at least 150%, even more preferably at least 165% of the ripening period and/or post-harvest shelf life of a tomato fruit being homozygous for the wild type Rin allele. In yet another aspect, the ripening period and/or post-harvest shelf life is at least 180%, more preferably at least 200% even more preferably at least 250% of the ripening period and/or post-harvest shelf life of a tomato fruit being homozygous for the wild type Rin allele.

In still another aspect of the invention tomato plants are provided that have the same or similar delayed ripening and/or increased shelf life as tomato plants of the invention, of which representative seeds were deposited by Nunhems B.V. and accepted for deposit on 27 Feb. 2012 at the NCIMB Ltd. (Ferguson Building, Craibstone Estate, Bucksburn Aberdeen, Scotland AB21 9YA, UK) according to the Budapest Treaty, under the Expert Solution (EPC 2000, Rule 32(1)). Seeds were given the following deposit numbers: NCIMB 41937 (mutant 2558), NCIMB 41938 (mutant 5225), and NCIMB 41939 (mutant 5996).

According to a further aspect the invention provides a cell culture or tissue culture of the tomato plant of the invention. The cell culture or tissue culture comprises regenerable cells. Such cells can be derived from leaves, pollen, embryos, cotyledon, hypocotyls, meristematic cells, roots, root tips, anthers, flowers, seeds and stems.

Seeds from which plants according to the invention can be grown are also provided, as well as packages containing such seeds. Also a vegetative propagations of plants according to the invention are an aspect encompassed herein. Likewise harvested fruits and fruit parts, either for fresh consumption or for processing or in processed form are encompassed. Fruits may be graded, sized and/or packaged. Fruits may be sliced or diced or further processed.

The invention also relates to food and/or food products incorporating the fruit or part of a fruit of a tomato plant of the invention. As used herein, food refers to nutrients consumed by human or animal species. Examples are sandwiches, salads, sauces, ketchup and the like.

In another aspect the invention relates to a method of producing a tomato plant of the invention comprising the steps of: (a)
a. obtaining plant material from a tomato plant;
b. treating said plant material with a mutagen to create mutagenized plant material;
c. analyzing said mutagenized plant material to identify a plant having at least one mutation in at least one rin allele having substantial sequence identity to SEQ ID NO: 1
   The method may further comprise analyzing the ripening period and/or shelf life of tomato fruits of the selected plant or progeny of the plant and selecting a plant of which the fruit have delayed ripening and/or extended shelf-life.
   In one aspect the mutation may be selected from a mutation in the K-domain of the rin protein. In one aspect the mutation is selected from the group consisting of T3949C, G3692A and G2652A of SEQ ID NO: 9. In this method, the plant material of step a) is preferably selected from the group consisting of seeds, pollen, plant cells, or plant tissue of a tomato plant line or cultivar. Plant seeds being more preferred. In another aspect, the mutagen used in this method is ethyl methanesulfonate. In step b) and step c) the mutagenized plant material is preferably a mutant population, such as a tomato TILLING population.
   Thus, in one aspect a method for producing a tomato plant comprising delayed fruit ripening and/or longer fruit shelf-life is provided comprising the steps of:
a) providing a tomato TILLING population,
b) screening said TILLING population for mutants in the rin gene, especially in the K-domain encoding nucleotide sequence, and
c) selecting from the mutant plants of b) those plants (or progeny of those plants) of which the fruits have a delayed ripening and/or longer shelf life than wild type (Rin/Rin) fruits.

Mutant plants (M1) are preferably selfed one or more times to generate for example M2 populations or preferably M3 or M4 populations for phenotyping. In M2 populations the mutant allele is present in a ratio of 1 (homozygous for mutant allele):2 (heterozygous for mutant allele):1 (homozygous for wild type allele).

In yet a further aspect the invention relates to a method for producing a hybrid Solanum lycopersicum plant, said method comprising:
(a) obtaining a first Solanum lycopersicum plant of the current invention and
(b) crossing said first Solanum lycopersicum plant with a second Solanum lycopersicum plant;
   wherein said hybrid Solanum lycopersicum plant comprises a rin allele having one or more mutations wherein said mutations result in production of a mutant rin protein having reduced activity compared to wild type Rin protein.

Plants and plant parts (e.g. fruits, cells, etc.) of the invention can homozygous or heterozygous for the mutant rin allele.

Preferably the plants according to the invention, which comprise one or more mutant rin alleles (or variants), and which produce a mutant rin protein having reduced activity compared to wild type Rin protein, do not produce fewer fruits than the wild type plants. Thus, fruit number per plant is preferably not reduced.

Other putative RIN genes/proteins can be identified in silico, e.g. by identifying nucleic acid or protein sequences in existing nucleic acid or protein database (e.g. GENBANK, SWISSPROT, TrEMBL) and using standard sequence analysis software, such as sequence similarity search tools (BLASTN, BLASTP, BLASTX, TBLAST, FASTA, etc.).

In one embodiment reduced-function mutant rin proteins (including variants or orthologs, such as rin proteins of wild tomato relatives) are provided and plants and plant parts comprising one or more rin alleles in their genome, which encode reduced-function mutants, whereby the reduced-function confers slower fruit ripening or/or a longer shelf life compared to Solanum lycopersicum being homozygous for the wild type Rin allele.

In another aspect the tomato plant of the invention comprises a mc allele which is optionally identical or essentially identical to a mc allele in a wild type plant.

In a further aspect the tomato plant of the invention produces MC protein or functional variants thereof having at least 85% or 90%, or 93%, or 97% or 99%, or 99.5%, or 99.9% sequence identity to wild type MC protein as defined in NCBI Solanum lycopersicum MADS-box transcription factor MADS-MC, mRNA, accession number 001247736 (http://www.ncbi.nlm.nih.gov/nuccore/NM_{—}001247736).

In another aspect, the invention relates to a tomato plant of the invention having an endogenous rin allele, in homozygous or heterozygous form, encoding a loss-of-function rin protein or reduced-function rin protein, said rin protein having substantial sequence identity to SEQ. ID NO: 2 or being 100% identical to the protein of SEQ ID NO: 2.

In another aspect, the invention relates to a tomato plant of the invention having an endogenous rin allele, in homozygous or heterozygous form, encoding a loss-of-function rin protein or reduced-function rin protein, said rin protein having substantial sequence identity to SEQ. ID NO: 3 or being 100% identical to the protein of SEQ ID NO: 3.

In another aspect, the invention relates to a tomato plant of the invention having an endogenous rin allele, in homozygous or heterozygous form, encoding a loss-of-function rin protein or reduced-function rin protein, said rin protein having substantial sequence identity to SEQ. ID NO: 4 or being 100% identical to the protein of SEQ ID NO: 4.

In another embodiment the invention relates to an isolated protein having substantial sequence identity to SEQ. ID NO: 2 or 100% sequence identity to SEQ. ID NO: 2. In still a further embodiment, the invention relates to an isolated nucleic acid sequence encoding a protein having substantial sequence identity to SEQ. ID NO: 2 or 100% sequence identity to SEQ. ID NO: 2.

In another embodiment the invention relates to an isolated protein having substantial sequence identity to SEQ. ID NO: 3 or 100% sequence identity to SEQ. ID NO: 3. In still a further embodiment, the invention relates to an isolated nucleic acid sequence encoding a protein having substantial sequence identity to SEQ. ID NO: 3 or 100% sequence identity to SEQ. ID NO: 3.

In another embodiment the invention relates to an isolated protein having substantial sequence identity to SEQ. ID NO: 2 or 100% sequence identity to SEQ. ID NO: 4. In still a further embodiment, the invention relates to an isolated nucleic acid sequence encoding a protein having substantial sequence identity to SEQ. ID NO: 2 or 100% sequence identity to SEQ. ID NO: 4.

In an even further embodiment, the invention relates to an isolated nucleic acid sequence, DNA or RNA, having substantial sequence identity to SEQ. ID NO: 6 or having 100% sequence identity to SEQ. ID NO: 6; or to an isolated nucleic acid sequence which is being transcribed into a nucleic acid sequence having substantial sequence identity to SEQ. ID NO: 6 or having 100% sequence identity to SEQ. ID NO: 6.

In an even further embodiment, the invention relates to an isolated nucleic acid sequence, DNA or RNA, having substantial sequence identity to SEQ. ID NO: 7 or having 100% sequence identity to SEQ. ID NO: 7; or to an isolated nucleic acid sequence which is being transcribed into a nucleic acid sequence having substantial sequence identity to SEQ. ID NO: 7 or having 100% sequence identity to SEQ. ID NO: 7.

In an even further embodiment, the invention relates to an isolated nucleic acid sequence, DNA or RNA, having substantial sequence identity to SEQ. ID NO: 8 or having 100% sequence identity to SEQ. ID NO: 8; or to an isolated nucleic acid sequence which is being transcribed into a nucleic acid sequence having substantial sequence identity to SEQ. ID NO: 8 or having 100% sequence identity to SEQ. ID NO: 8.

Any type of mutation may lead to a reduction in function of the encoded Rin protein, e.g. insertion, deletion and/or replacement of one or more nucleotides in the cDNA (SEQ ID NO: 5, or variants) or in the corresponding genomic Rin sequence (SEQ ID NO: 9, or variant). Especially in any of the 8 exon sequences and/or intron/exon boundaries of Rin proteins. In a preferred embodiment, a rin nucleic acid sequence capable of conferring slower fruit ripening and/or a longer shelf life compared to Solanum lycopersicum being homozygous for the wild type Rin allele, whereby the nucleic acid sequence encodes a reduced-function Rin protein due to one or more mutations outside the MADS box domain (i.e. no mutation in the first 61 amino acid-encoding part of the wild type allele).

The in vivo reduced-function of such proteins can be tested as described herein, by determining the effect this mutant allele has on ripening period and/or shelf life period. Plants comprising a nucleic acid sequence encoding such mutant reduced-function proteins and having a slower fruit ripening and/or a longer shelf life compared to Solanum lycopersicum being homozygous for the wild type Rin allele can for example be generated using e.g. mutagenesis and identified by TILLING or identified using EcoTILLING, as known in the art. Also transgenic methods can be used to test in vivo functionality of a mutant nth allele encoding a mutant rin protein. A mutant allele can be operably linked to a plant promoter and the chimeric gene can be introduced into a tomato plant by transformation. Regenerated plants (or progeny, e.g. obtained by selfing), can be tested for fruit ripening period and/or shelf life. For example a tomato plant comprising a non-functional rin allele, such as the prior art rin allele (rin/rin), can be transformed to test the functionality of the transgenic rin allele.

TILLING (Targeting Induced Local Lesions IN Genomes) is a general reverse genetic technique that uses traditional chemical mutagenesis methods to create libraries of mutagenized individuals that are later subjected to high throughput screens for the discovery of mutations. TILLING combines chemical mutagenesis with mutation screens of pooled PCR products, resulting in the isolation of mis-sense and non-sense mutant alleles of the targeted genes. Thus, TILLING uses traditional chemical mutagenesis (e.g. EMS or MNU mutagenesis) or other mutagenesis methods (e.g. radiation such as UV) followed by high-throughput screening for mutations in specific target genes, such as RIN according to the invention. 51 nucleases, such as CEL1 or ENDO1, are used to cleave heteroduplexes of mutant and wildtype target DNA and detection of cleavage products using e.g. electrophoresis such as a LI-COR gel analyzer system, see e.g. Henikoff et al. Plant Physiology 2004, 135: 630-636. TILLING hasapplied been in many plant species, such as tomato. (see http://tilling.ucdavis.edu/index.php/Tomato Tilling), rice (Till et al. 2007, BMC Plant Biol 7: 19), Arabidopsis (Till et al. 2006, Methods Mol Biol 323: 127-35), -Brassica, maize (Till et al. 2004, BMC Plant Biol 4: 12), etc. Also EcoTILLING, whereby mutants in natural populations are detected, has been widely used, see Till et al. 2006 (Nat Protoc 1: 2465-77) and Comai et al. 2004 (Plant J 37: 778-86).

In one embodiment of the invention (cDNA or genomic) nucleic acid sequences encoding such mutant rin proteins comprise one or more non-sense and/or mis-sense mutations, e.g. transitions (replacement of purine with another purine (A⇄G) or pyrimidine with another pyrimidine (C⇄T)) or transversions (replacement of purine with pyrimidine, or vice versa (C/T⇄A/G). In one embodiment the non-sense and/or mis-sense mutation(s) is/are in the nucleotide sequence encoding any of the Rin exons, more preferably outside the MADS-domain regions or an essentially similar domain of a variant Rin protein, i.e. in a domain comprising at least 80%, 90%, 95%, 98%, 99% amino acid identity to amino acids 1 to 61 of SEQ ID NO: 1.

In one embodiment a rin nucleotide sequence comprising one or more non-sense and/or mis-sense mutations in the exon 2-, exon 3-, exon 4-, exon 5-, exon 6-, exon 7- and/or exon 8-encoding sequence are provided, as well as a plant comprising such a mutant allele resulting in delayed fruit ripening and/or a longer shelf life compared to Solanum lycopersicum being homozygous for the wild type Rin allele.

In a specific embodiment of the invention tomato plants and plant parts (fruits, seeds, etc.) comprising a mutant reduced-function rin allele are provided.

In one embodiment, the reduced-function rin protein is a truncated protein, i.e. a protein fragment of any one of the Rin proteins defined further above (including variants thereof). In general EMS (Ethyl methanesulfonate) induces substitutions of guanine/cytosine to adenin/thymine. In case of a glutamine (Gln or Q, encoded by the nucleotides CAA or CAG) or arginine (Arg or R, encoded by the nucleotides CGA) codon, a substitution of the cytosine for thymine can lead to the introduction of a stop codon in the reading frame (for example CAA/CAG/CGA to TAA/TAG/TGA) resulting in a truncated protein.

Also provided are nucleic acid sequences (genomic DNA, cDNA, RNA) encoding reduced-function rin proteins, such as for example rin depicted in SEQ ID NO: 2, 3 or 4; or variants thereof as defined above (including any chimeric or hybrid proteins or mutated proteins or truncated proteins). Due to the degeneracy of the genetic code various nucleic acid sequences may encode the same amino acid sequence. The nucleic acid sequences provided include naturally occurring, artificial or synthetic nucleic acid sequences. A nucleic acid sequence encoding Rin is provided for in SEQ ID NO: 5 (wild type cDNA), sequence of cultivar Ailsa Craig, Science 2002, vol 296, pp 343, Genbank AF448522; and SEQ ID NO: 9 (genomic sequence of tomato cv Heinz 1706, with introns and exons as described before).

It is understood that when sequences are depicted in as DNA sequences while RNA is referred to, the actual base sequence of the RNA molecule is identical with the difference that thymine (T) is replace by uracil (U). When referring herein to nucleotide sequences (e.g DNA or RNA) italics are used, e.g. rin allele, while when referring to proteins, no italics are used, e.g. rin protein. Mutants are in small letters (e.g rin allele or rin protein), while wild type/functional forms start with a capital letter (Rin allele or Rin protein).

Also provided are nucleic acid sequences (genomic DNA, cDNA, RNA) encoding mutant rin proteins, i.e. reduced function rin proteins, as described above, and plants and plant parts comprising such mutant sequences. For example, rin nucleic acid sequences comprising one or more non-sense and/or mis-sense mutations in the wild type Rin coding sequence, rendering the encoded protein having a reduced function in vivo. Also sequences with other mutations are provided, such as splice-site mutants, i.e. mutations in the genomic rin sequence leading to aberrant splicing of the pre-mRNA, and/or frame-shift mutations, and/or insertions (e.g. transposon insertions) and/or deletions of one or more nucleic acids.

It is clear that many methods can be used to identify, synthesise or isolate variants or fragments of rin nucleic acid sequences, such as nucleic acid hybridization, PCR technology, in silico analysis and nucleic acid synthesis, and the like. Variants of SEQ ID NO: 9, may either encode wild type, functional Rin proteins, or they may encode reduced-function mutant alleles of any of these, as for example generated e.g. by mutagenesis and/or identified by methods such as TILLING or EcoTILLING, or other methods.

A plant of the invention can be used in a conventional plant breeding scheme to produce more plants with the same characteristics or to introduce the mutated rin allele into other plant lines or varieties of the same or related plant species.

Also transgenic plants can be made using the mutant rin nucleotide sequences of the invention using known plant transformation and regeneration techniques in the art. An “elite event” can be selected, which is a transformation event having the chimeric gene (comprising a promoter operably linked to a nucleotide sequence encoding a reduced-function rin protein) inserted in a particular location in the genome, which results in good expression of the desired phenotype.

The plants of the invention as described above are homozygous for the mutant rin allele, or heterozygous. To generate plants comprising the mutant allele in homozygous form, selfing can be used.

The mutant rin alleles according to the invention can be transferred to any other tomato plant by traditional breeding techniques, such as crossing, selfing, backcrossing, etc. Thus any type of tomato having delayed ripening and/or longer shelf life due to the presence of at least one mutant rin allele according to the invention can be generated. Any S. lycopersicum may be generated and/or identified having at least one mutant rin allele in its genome and producing a rin protein having reduced activity compared to wild type Rin protein. The tomato plant may, thus, be any cultivated tomato, any commercial variety, any breeding line or other, it may be determinate or indeterminate, open pollinated or hybrid, producing fruits of any color, shape and size. The mutant allele generated and/or identified in a particular tomato plant, or in a sexually compatible relative of tomato, may be easily transferred into any other tomato plant by breeding (crossing with a plant comprising the mutant allele and then selecting progeny comprising the mutant allele).

The presence or absence of a mutant rin allele according to the invention in any tomato plant or plant part and/or the inheritance of the allele to progeny plants can be determined phenotypically and/or using molecular tools (e.g. detecting the presence or absence of the rin nucleotide or rin protein using direct or indirect methods).

The mutant allele is in one embodiment generated or identified in a cultivated plant, but may also be generated and/or identified in a wild plant or non-cultivated plant and then transferred into an cultivated plant using e.g. crossing and selection (optionally using interspecific crosses with e.g. embryo rescue to transfer the mutant allele). Thus, a mutant rin allele may be generated (human induced mutation using mutagenesis techniques to mutagenize the target rin gene or variant thereof) and/or identified (spontaneous or natural allelic variation) in Solanum lycopersicum or in other Solanum species include for example wild relatives of tomato, such as S. cheesmanii, S. chilense, S. habrochaites (L. hirsutum), S. chmielewskii, S. lycopersicum×S. peruvianum, S. glandulosum, S. hirsutum, S. minutum, S. parviflorum, S. pennellii, S. peruvianum, S. peruvianum var. humifusum and S. pimpinellifolium, and then transferred into a cultivated Solanum plant, e.g. Solanum lycopersicum by traditional breeding techniques. The term “traditional breeding techniques” encompasses herein crossing, selfing, selection, double haploid production, embryo rescue, protoplast fusion, transfer via bridge species, etc. as known to the breeder, i.e. methods other than genetic modification by which alleles can be transferred.

In another embodiment, the plant comprising the mutant rin allele (e.g. tomato) is crossed with another plant of the same species or of a closely related species, to generate a hybrid plant (hybrid seed) comprising the mutant rin allele. Such a hybrid plant is also an embodiment of the invention.

In one embodiment F1 hybrid tomato seeds (i.e. seeds from which F1 hybrid tomato plants can be grown) are provided, comprising at least one rin allele according to the invention. F1 hybrid seeds are seeds harvested from a cross between two inbred tomato parent plants. Such an F1 hybrid may comprise one or two mutant rin alleles according to the invention. Thus, in one embodiment a plant according to the invention is used as a parent plant to produce an F1 hybrid, the fruit of which have delayed ripening and/or longer shelf-life than wild type Rin/Rin plants.

Also a method for transferring a mutant rin allele to another plant is provided, comprising providing a plant comprising a mutant rin allele in its genome, whereby the mutant allele produce fruits that show slower fruit ripening and/or a longer shelf life compared to Solanum lycopersicum being homozygous for the wild type Rin allele (as described above), crossing said plant with another plant and obtaining the seeds of said cross. Optionally plants obtained from these seeds may be further selfed and/or crossed and progeny selected comprising the mutant allele and producing fruits with delayed ripening and/or longer shelf-life due to the presence of the mutant allele compared to plants comprising the wild type Rin allele.

As mentioned, it is understood that other mutagenesis and/or selection methods may equally be used to generate mutant plants according to the invention. Seeds may for example be radiated or chemically treated to generate mutant populations. Also direct gene sequencing of rin may be used to screen mutagenized plant populations for mutant alleles. For example KeyPoint screening is a sequence based method which can be used to identify plants comprising mutant rin alleles (Rigola et al. PloS One, March 2009, Vol 4(3):e4761).

Thus, non-transgenic mutant tomato plants which produce lower levels of wild type Rin protein in fruits are provided, or which completely lack wild type Rin protein in fruits, and which produce reduced-function rin protein in fruits due to one or more mutations in one or more endogenous rin alleles, are provided. These mutants may be generated by mutagenesis methods, such as TILLING or variants thereof, or they may be identified by EcoTILLING or by any other method. Rin alleles encoding reduced-functional rin protein may be isolated and sequenced or may be transferred to other plants by traditional breeding methods.

Any part of the plant, or of the progeny thereof, is provided, including harvested fruit, harvested tissues or organs, seeds, pollen, flowers, ovaries, etc. comprising a mutant rin allele according to the invention in the genome. Also plant cell cultures or plant tissue cultures comprising in their genome a mutant rin allele are provided. Preferably, the plant cell cultures or plant tissue cultures can be regenerated into whole plants comprising a mutant rin allele in its genome. Also double haploid plants (and seeds from which double haploid plants can be grown), generated by chromosome doubling of haploid cells comprising an rin mutant allele, and hybrid plants (and seeds from which hybrid plants can be grown) comprising a mutant rin allele in their genome are encompassed herein, whereby the double haploid plants and hybrid plants produce delayed ripening and/or longer shelf life fruits according to the invention.

Preferably, the mutant plants also have good other agronomic characteristics, i.e. they do not have reduced fruit numbers and/or reduced fruit quality compared to wild type plants. In a preferred embodiment the plant is a tomato plant and the fruit is a tomato fruit, such as a processing tomato, fresh market tomato of any shape or size or colour. Thus, also harvested products of plants or plant parts comprising one or two mutant rin alleles are provided. This includes downstream processed products, such as tomato paste, ketchup, tomato juice, cut tomato fruit, canned fruit, dried fruit, peeled fruit, etc. The products can be identified by comprising the mutant allele in their genomic DNA.

### Seed Deposits

A representative sample of seeds of three tomato TILLING mutants according to Example 1, were deposited by Nunhems B.V. and accepted for deposit on 27 Feb. 2012 at the NCIMB Ltd. (Ferguson Building, Craibstone Estate, Bucksburn Aberdeen, Scotland AB21 9YA, UK) according to the Budapest Treaty, under the Expert Solution (EPC 2000, Rule 32(1)). Seeds were given the following deposit numbers: NCIMB 41937 (mutant 2558), NCIMB 41938 (mutant 5225), and NCIMB 41939 (mutant 5996).

The Applicant requests that samples of the biological material and any material derived therefrom be only released to a designated Expert in accordance with Rule 32(1) EPC or related legislation of countries or treaties having similar rules and regulation, until the mention of the grant of the patent, or for 20 years from the date of filing if the application is refused, withdrawn or deemed to be withdrawn.

Access to the deposit will be available during the pendency of this application to persons determined by the Director of the U.S. Patent Office to be entitled thereto upon request. Subject to 37 C.F.R. §1.808(b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer, and will be replaced if it ever becomes nonviable during that period. Applicant does not waive any rights granted under this patent on this application or under the Plant Variety Protection Act (7 USC 2321 et seq.).

### EXAMPLES

### General Methods

PCR amplification products were directly sequenced by a service company (BaseClear, The Netherlands, http://www.baseclear.com/) using the same primers as were used for the amplification. The obtained sequences were aligned using a computer program (CLC Bio Main Work Bench, Denmark, www.cicbio.com) to identify the nucleotide changes.

### Materials

Water used for analyses and mutagenis is tap water filtered in an Milli-Q water Integral system, Milli-Q type Reference A+ supplied with a Q-gard T2 Cartridge and a Quantum TEX Cartridge. Water resistance is >=18 MOhm.

Ethyl Methanesulfonate (EMS) (pure) was obtained from Sigma, product number M0880.

Measurement of Tomato Ripening and/or Shelf-Life Time or Periods

Tomato ripening and/or shelf life time or periods can be measured by various methods known in the art like for example making periodically visual assessments of fruits and/or measurement of fruit firmness or softening, measurement of lycopene contents in the tomato fruits, ethylene production by the fruits, colour of the fruits or any alternative method or combination of methods. Fruit firmness can for example be measured by evaluating resistance to deformation in units of for example 0.1 mm as measured with a penetrometer fitted with a suitable probe (e.g. a probe of 3 mm) (Mutschler et al, 1992, Horscience 27 pp 352-355) (Marinez et at 1995 Acta Horticulturae 412 pp 463-469). Alternative methods exist in the art, such as use of a texturometer (Bui et al. 2010; International Journal of Food Properties, Volume 13, Issue 4). For example an Instron 3342 Single Column Testing System can be suitably used.

Fruit colour can be classified by the U.S. standards for grades of fresh tomato (U.S. Dept of Agriculture, 1973, US standards for grades of fresh tomatoes, U.S. Dept Agr. Agr. Mktg. Serv., Washington D.C.), measuring the colour with a chromometer (Mutschler et al, 1992, Horscience 27 pp 352-355) or by comparing the colour to a colour chart like the Royal Horticultural Society (RHS) Color Chart (www.rhs.org.uk).

Lycopene content can be determined according to the reduced volumes of organic solvents method of Fish et al. A quantitative assay for lycopene that utilizes reduced volumes of organic solvents.

J. Food Compos. Anal. 2002, 15, 309-317. This method can be used to determine lycopene content measured directly on intact tomato fruit while simultaneously estimating the basic physicochemical characteristics: color, firmness, soluble solids, acidity, and pH (Clement et al, J. Agric. Food Chem. 2008, 56, 9813-9818).

Ethylene release can be measured by placing the fruit in a closed space, e.g. in a 0.5 l glass holder. One ml of holder atmosphere can be extracted after one hour and amount of ethylene gas produced can be quantified using a gas chromatograph (e.g. a Hewlett-Packard 5890) equipped with a suitable detection unit, e.g. a flame ionisation detector, and a suitable column (e.g. a 3 m stainless steel column with an inner diameter of 3.5 mm containing activated alumina of 80/100 mesh). Ethylene production can be expressed as the amount in n1 of ethylene given off per gram of fruit per hour (n1 g⁻¹ h⁻¹) (Marinez et at 1995 Acta Horticulturae 412 pp 463-469).

Alternatively, ethylene production can be measured as described further below, using real-time measurements with a laser-based ethylene detector (ETD-300, Sensor Sense B.V., Nijmegen, the Netherlands) in combination with a gas handling system (Cristecu et al., 2008).

### Example 1

### Mutagenesis

A highly homozygous inbred line used in commercial processing tomato breeding was used for mutagenesis treatment with the following protocol. After seed germination on damp Whatman® paper for 24 h, −20,000 seeds, divided in 8 batches of 2500 respectively, were soaked in 100 ml of ultrapure water and ethyl methanesulfonate (EMS) at a concentration of 1% in conical flasks. The flasks were gently shaken for 16 h at room temperature. Finally, EMS was rinsed out under flowing water. Following EMS treatment, seeds were directly sown in the greenhouse. Out of the 60% of the seeds that germinated, 10600 plantlets were transplanted in the field. From these 10600 plantlets, 1790 were either sterile or died before producing fruit. For each remaining M1 mutant plant one fruits was harvested and its seeds isolated. The obtained population, named M2 population, is composed of 8810 seeds lots each representing one M2 family. Of these, 585 families were excluded from the population due to low seed availability.

DNA was extracted from a pool of 10 seeds originating from each M2 seed lot. Per mutant line, 10 seeds were pooled in a Micronic® deepwell tube; http://www.micronic.com from a 96 deep-well plate, 2 stainless balls were added to each tube. The tubes and seeds were frozen in liquid nitrogen for 1 minute and seeds were immediately ground to a fine powder in a Deepwell shaker (Vaskon 96 grinder, Belgium; http://www.vaskon.com) for 2 minutes at 16.8 Hz (80% of the maximum speed). 300 μl Agowa® Lysis buffer P from the AGOWA® Plant DNA Isolation Kit http://www.agowa.de was added the sample plate and the powder was suspended in solution by shaking 1 minute at 16.8 Hz in the Deepwell shaker. Plates were centrifuged for 10 minutes at 4000 rpm. 75 μl of the supernatant was pipetted out to a 96 Kingfisher plate using a Janus MDT® (Perkin Elmer, USA; http://www.perkinelmer.com) platform (96 head). The following steps were performed using a Perkin Elmer Janus® liquid handler robot and a 96 Kingfisher® (Thermo labsystems, Finland; http://www.thermo.com). The supernatant containing the DNA was diluted with binding buffer (150 μl) and magnetic beads (20 μl). Once DNA was bound to the beads, two successive washing steps were carried out (Wash buffer 1: Agowa wash buffer 1⅓, ethanol ⅓, isopropanol ⅓; Wash buffer 2: 70% ethanol, 30% Agowa wash buffer 2) and finally eluted in elution buffer (100 μl MQ, 0.025 μl Tween).

Grinding ten S. lycopersicum seeds produced enough DNA to saturate the magnetic beads, thus highly homogenous and comparable DNA concentrations of all samples were obtained. Comparing with lambda DNA references, a concentration of 30 ng/μl for each sample was estimated. Two ti diluted DNA was 4 fold flat pooled. 2 μl pooled DNA was used in multiplex PCRs for mutation detection

Primers used to amplify gene fragments for HRM were designed using a computer program (Primer3, http://primer3.sourceforge.net/). The length of the amplification product was limited between 200 and 400 base pairs. Quality of the primers was determined by a test PCR reaction that should yield a single product.

Polymerase Chain Reaction (PCR) to amplify gene fragments. 10 ng of genomic DNA mixed with 4 μl reaction buffer (5× Reaction Buffer), 2 μl 10×LC dye ((LCGreen+ dye, Idaho Technology Inc., UT, USA), 5 pmole of forward and reverse primers each, 4 nmole dNTPs (Life Technologies, NY, USA) and 1 unit DNA polymerase (Hot Start II DNA Polymerase) in a total volume of 10 μl. Reaction conditions were: 30 s 98° C., then 40 cycles of 10 s. 98° C., 15 s 60° C., 25 s of 72° C. and finally 60 s at 72° C.

High Resolution Melt curve analysis (HRM) has been proven to be sensitive and high-throughput methods in human and plant genetics. HRM is a non-enzymatic screening technique. During the PCR amplification dye (LCGreen+ dye, Idaho Technology Inc., UT, USA) molecules intercalate between each annealed base pair of the double stranded DNA molecule. When captured in the molecule, the dye emits fluorescence at 510 nm after excitation at 470 nm. A camera in a fluorescence detector (LightScanner, Idaho Technology Inc., UT, USA) records the fluorescence intensity while the DNA sample is progressively heated. At a temperature dependent on the sequence specific stability of the DNA helices, the double stranded PCR product starts to melt, releasing the dye. The release of dye results in decreased fluorescence that is recorded as a melting curve by the fluorescence detector. Pools containing a mutation form hetero duplexes in the post-PCR fragment mix. These are identified as differential melting temperature curves in comparison to homo duplexes.

Mutants showing a delayed ripening were selected and the type of mutation in the rin gene was determined.

The presence of the particular mutation in individual plants was confirmed repeating the HRM analysis on DNA from the individual M2 seed lots of the identified corresponding DNA pool. When the presence of the mutation, based on the HRM profile, was confirmed in one of the four individual M2 family DNA samples, the PCR fragments were sequenced to identify the mutation in the gene.

Once the mutation was known the effect of such an mutation was predicted using a computer program CODDLe (for Choosing codons to Optimize Discovery of Deleterious Lesions, http://www.proweb.org/coddle/) that identifies the region(s) of a user-selected gene and of its coding sequence where the anticipated point mutations are most likely to result in deleterious effects on the gene's function.

Seeds from M2 families that contain mutations with predicted effect on protein activity were sown for phenotypic analysis of the plants.

Homozygous mutants were selected or obtained after selfing and subsequent selection. The effect of the mutation on the corresponding protein and phenotype of the plant was determined.

Seeds containing the different identified mutations were germinated and plants were grown in pots with soil the greenhouse with 16/8 light dark regime and 18° C. night and 22-25° C. day temperature. For each genotype 5 plants were raised. The second, third and fourth inflorescence were used for the analysis. The inflorescences were pruned remaining six flowers per inflorescence that were allowed to set fruit by self-pollination. The dates of fruit set of the first and sixth flower was recorded as was the date of breaker and red stage of the first and sixth fruit. At red stage of the fourth fruit the truss was harvested and stored in an open box in the greenhouse. Fruit condition of the fruits was recorded during the whole ripening period by making pictures from each truss. After harvest pictures were made per box containing all trusses from one genotype.

At later stages fruit condition was determined based on visual assessment of the fruits and the date when the oldest fruit became ‘bad’ was recorded and further fruit deterioration was recorded (indicated by further fruit softness assessed by pinching the fruits, and visual assessment of dehydration/water loss, breaking of the skin and fungal growth).

The following mutants were identified: mutant 5996, mutant 5225, and mutant 2558 and seeds were deposited at the NCIMB under the Accession numbers given below.

Mutant 5996 (NCIMB 41939)

Nucleotide 3949 is changed from a T to C at (SEQ ID NO: 9), counting A in the ATG of the START CODON as nucleotide position 1. This causes a T to C at nucleotide 335 of SEQ ID NO: 5, again counting A in the ATG of the START CODON as nucleotide position 1. This mutation results in a change from leucine to proline at amino acid 112 in the expressed protein. The L112P mutation is within the K-domain of the RIN protein. The protein sequence of mutant 5996 is depicted in SEQ ID NO: 4. The corresponding cDNA is depicted in SEQ ID NO: 8.

Mutant 5225 (NCIMB 41938)

correlated with a G to A at nucleotide 3692 of SEQ ID NO: 9 counting A in the ATG of the START CODON as nucleotide position 1. This causes a G to A at nucleotide 304 of SEQ ID NO: 5, again counting A in the ATG of the START CODON as nucleotide position 1. This mutation results in a change from glutamic acid to lysine at amino acid 102 in the expressed protein. The E102K mutation is within the K-domain of the Rin protein. The protein sequence of mutant 5225 is depicted in SEQ ID NO: 3. The corresponding cDNA is depicted in SEQ ID NO: 7.

Mutant 2558 (NCIMB 41937)

correlated with a change of G to A at nucleotide 3652 of SEQ ID NO: 9 (mutant 2558) counting A in the ATG of the START CODON as nucleotide position 1. Mutant 2558 carries a mutation in the last nucleotide before the splicing acceptor side between intron 2 and exon 3. Such a mutation close to a splicing site may cause mis-splicing. In this case, as it is just before the beginning of exon 3, it was expected that the corresponding cDNA (SEQ ID NO: 6) lacks exon 3 will cause a shift in the reading frame of exon 4, which leads to a stop codon 4 amino acid after the mutation. The truncated protein still contains the complete MADS-domain but lost the entire K-box domain, see SEQ ID NO: 2.

Plants comprising mutations in the target sequence, such as the above mutant plants or plants derived therefrom (e.g. by selfing or crossing) and comprising the mutant rin allele, were screened phenotypically for their fruit ripening and shelf live.

### Example 2

### Ripening Behaviour of the Rin Mutants

Seeds containing the different mutations were germinated and plants were grown in pots with soil the greenhouse with 16/8 light dark regime and 18° C. night and 22-25° C. day temperature. For each genotype 5 plants were raised. The second, third and fourth inflorescence were used for the analysis. The inflorescences were pruned, leaving six flowers per inflorescence that were allowed to set fruit by self-pollination. The dates of fruit set of the first and sixth flower was recorded as was the date of breaker and red stage of the first and sixth fruit. At red stage of the 4^{th} fruit the truss was harvested and stored in an open box in the greenhouse. Fruit condition of the fruits was recorded during the whole ripening period by making pictures from each truss. After harvest pictures were made per box containing all trusses from one genotype.

At later stages fruit condition was determined based on visual assessment of the fruits and the date when the oldest fruit became ‘bad’ was recorded and further fruit deterioration was recorded (indicated by further fruit softness assessed by pinching the fruits, and visual assessment of dehydration/water loss, breaking of the skin and fungal growth).

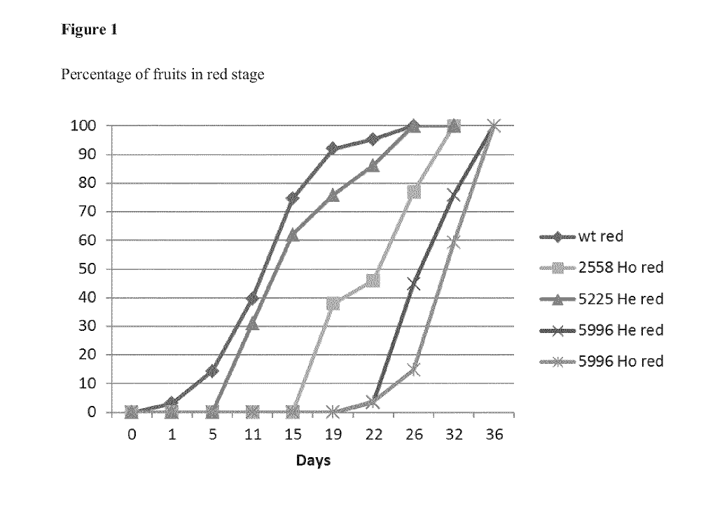

The ripening behaviour of the fruits is shown in FIG. 1. All mutants show a delay in ripening, i.e. fruits of the mutants require more days to become red. Especially mutant 2558 and 5996 show a significant delay of several days.

A characteristic of fruits of the plants of the invention is that breaker stage starts later and fruits reach the red stage later than wild type fruits. Post-harvest characteristics are shown below:

The day on which the first fruit of the wild type (Rin/Rin) plant came into breaker stage was taken as day 1. The days thereafter were numbered as consecutive days.

| | First | All fruits | First fruit | 100% fruit | First fruits |
|---|---|---|---|---|---|
| | fruit in | in breaker | in red | in red | in ‘bad’ |
| | Breaker | stage on | stage on | stage on | stage on |
| | on day no. | day no. | day no. | day no. | day no. |
| Wt | 1 | 25 | 2 | 27 | 37 |
| 5996 He | 16 | 35 | 23 | 37 | >49 |
| 5996 Ho | 12 | 37 | 23 | 37 | >49 |

n.d. = not determined

As can be seen, mutant fruits enter breaker stage later and the date when all fruits are in breaker stage is also later. Equally, mutant fruits come into the red stage later and the date when all fruits of a mutant line are in red stage is also significantly later than for the wild type.
For mutant 5996 it took more than 49 days before the first fruit became bad, and unsuitable for consumption or sale, i.e. at least 12 days longer than for the wild type fruits.

### Example 3

### Ethylene Release

Ethylene released by tomato fruits was measured in real-time with a laser-based ethylene detector (ETD-300, Sensor Sense B.V., Nijmegen, the Netherlands) in combination with a gas handling system (Cristecu et al., Laser-based systems for trace gas detection in life sciences. Appl Phys B 2008; 92 pp 343-9). Six glass cuvettes (100 mL volume) were used per experiment, one as a reference without plant material. Air was sampled from the lab and passed through a platinum based catalyzer (Sensor Sense B.V., Nijmegen, the Netherlands) to remove traces of ethylene or other hydrocarbons. Between the sample and the detector scrubbers with KOH and CaCl2 were placed to reduce the CO2 concentration (to less than 1 ppm) and decrease the water content in the gas flow, respectively.

Comparison of the ethylene released from fruits of mutant 2558 (homozygous for mutated rin allele) and 5996 (homozygous for mutated rin allele) with wild type (tapa, referring to line TPAADASU) at Pink stage and red stage revealed that at pink stage the ethylene production of both mutants 2558 and 5996 was significantly reduced compared to wild-type: <0.5 n1/(h·g) for the mutants versus 4.8 n1/(h·g) for the wild type. The difference at red stage is even more significant: <0.5 n1/(h·g) for the mutants versus 8.7 n1/(h·g) for the wild type. Wherein n1/(h·g) means nano liter per hour per gram of fruit.

### Example 4

### Real-Time Quantitative RT-PCR

Each tissue sample for the mature green (MG) and Breaker (BR) stages consisted of pieces from the pericarp tissue (0.5 cm*0.5 cm) from different fruits in triplicate, 5 different fruits per sample.

cDNA Synthesis

Total RNA was extracted with on-column DNase treatment (RNeasy; Qiagen) and quantified using a photospectrometer (Nanodrop 8000 Thermo Fisher Scientific Inc, USA). Half a microgram of RNA was used for reverse transcription to synthesize cDNA using a DNA removal and cDNAsynthesis kit (QuantiTect® reverse transcription kit, QIAGEN, Germany).

### Template Quantification

The cDNA equivalent of 5 ng of total RNA was used in a 20-μL PCR reaction on a Real-Time PCR System was used (Life Technologies Applied Biosystems, ViiA™ 7) with Power SYBER® Green PCR Master Mix (Applied Biosystems). In all experiments, three biological replicates of each sample type were tested. Absence of genomic DNA and primer dimers was confirmed by analysis of water control samples and by examination of dissociation curves. To normalize the qPCR data, three reference genes were used in each experiment (i.e. actin, ubiquitin, and SAND-family protein).

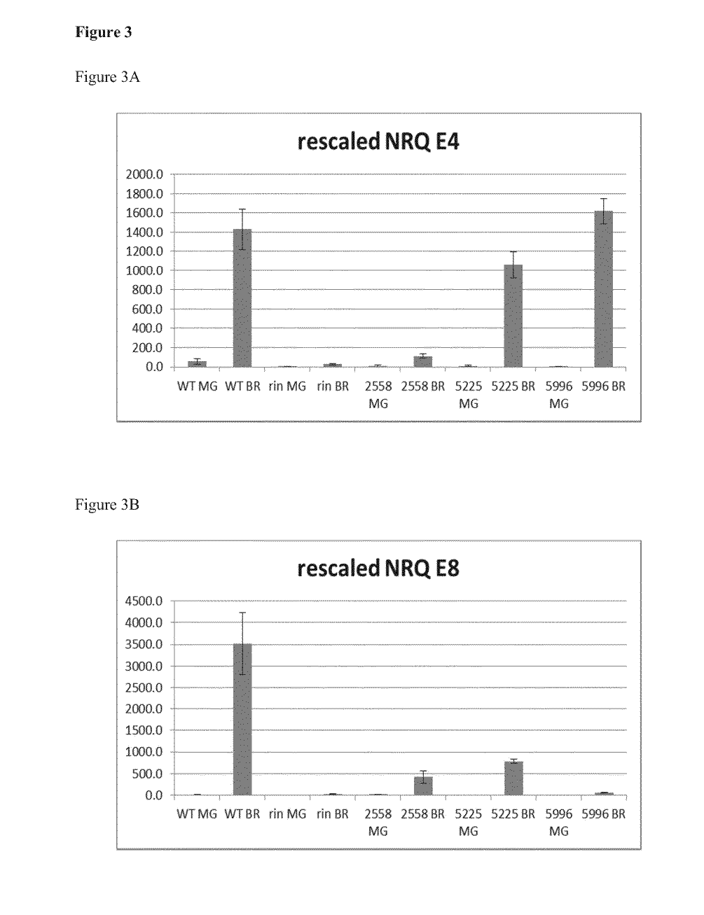

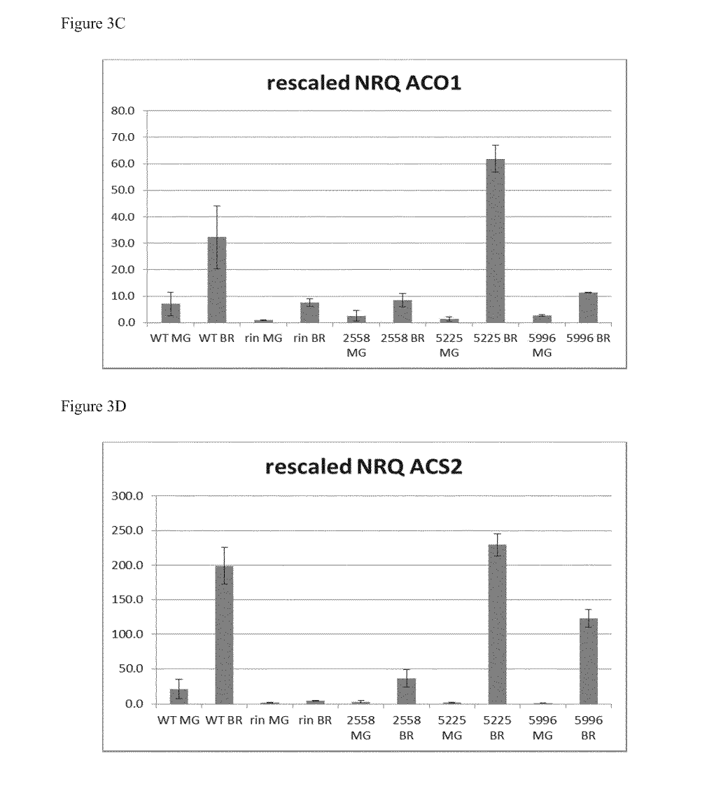

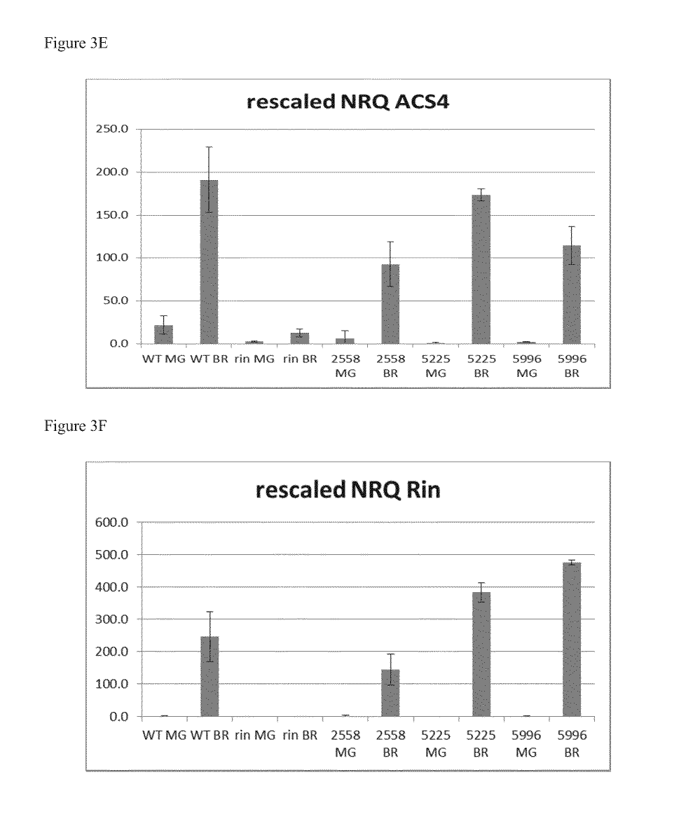

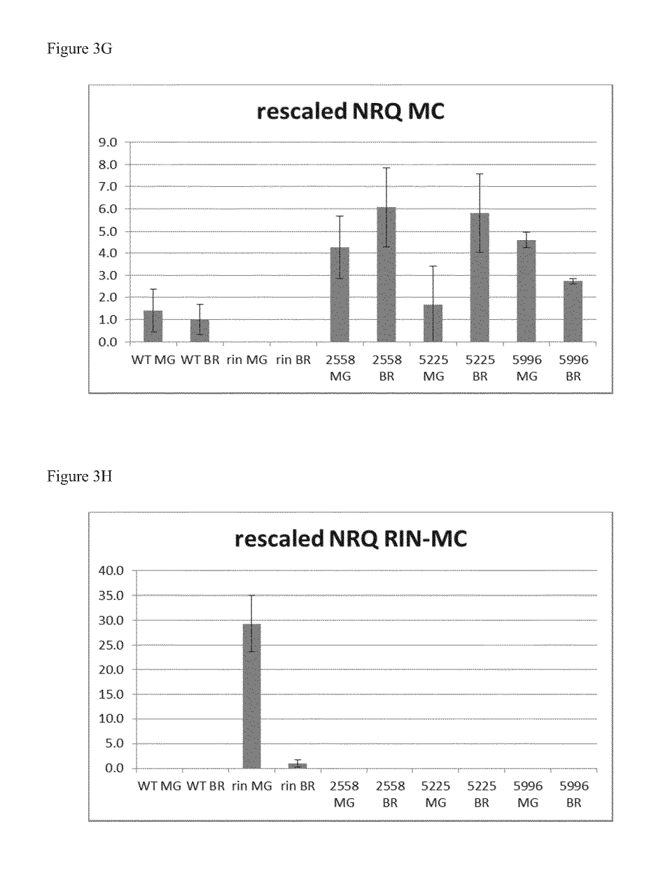

Quantitative PCR primers were designed using primer design software (CLC Genomic workbench, CLC Bio, USA) and are listed below. Relative quantity of template (RQ) were calculated as RQ=1/E^{Cq}; wherein E is the amplification efficiency (taken arbitrarily as 2); Cq is the number of cycles at a threshold level of fluorescence (quantification cycle or Cq. After that, the RQ of the gene of interest (GOI) was normalized for the total amount of cDNA to calculate: NRQ=(1/E^{Cq} GOI)/(1/E^{Cq} reference genes). The graphs in FIG. 3A-H present the NRQs after setting the lowest value to 1. The error bars represent the standard deviation between the biological replicates. The Student t-tests were calculated based in the logRQ values of the replicates. (Real time PCR data was interpreted as described in The Plant Cell April 2009 vol. 21 no. 4 pp 1031-1033;

Statistical differences were calculated using Student's t-test.

TABLE
Overview primers used for Real Time quantitative PCR.

| | | Forward primer | | Reverse primer | | |
|---|---|---|---|---|---|---|
| | | sequence 5′-end | | Sequence 5′-end | | GenBank |
| Figure | Primer | to 3′-end | Primer | to 3′-end | Gene | Acc. No. |
| 3D | 8114 | AAGCGCGATGAG | 8115 | AAAGTGGACGCAAAT | ACS2 | X59139² |
| | | GTTAGGTA | | CCATC | | |
| 3E | 8116 | AAATCTCCACCTT | 8117 | CCTAAGTCCTTGGAA | ACS4 | M88487² |
| | | CACTAACGAAC | | AGACTAGACAC | | |
| 3A | 8210 | AGAGGGTTGGAG | 8211 | AAAGGAGATTGGAAT | E4 | S44898² |
| | | GAGTAG | | ACGGG | | |
| 3B | 8120 | GCGGGGAGTCAT | 8121 | AACCGGGTGTAGGAG | E8 | X13437² |
| | | TAATAG | | GAA | | |
| 3C | 8122 | TGGAGATGAGAG | 8123 | TTCCATGGTTCACCAA | ACO1 | X58273¹ |
| | | AGCCAACA | | CTCA | | |
| 3G | 8279 | AGAGAAGAGGTG | 8280 | ATGCTTGTGGTTCCTT | LeMA | AF448521¹ |
| | | GATTAGTG | | TG | DS-MC | |
| 3F | 8283 | TTGTGGTGAGCA | 8284 | GCTGCATTTTCGGGTT | LeMA | AF448522¹ |
| | | AAGTGT | | GTA | DS-RIN | |
| 3H | 8283 | TTGTGGTGAGCA | 8280 | ATGCTTGTGGTTCCTT | rin | AF448523¹ |
| | | AAGTGT | | TG | RIN-MC | |
| | 3675 | CATTGTGCTCAGT | 3676 | TCTGCTGGAAGGTGCT | actin | BT013524⁴ |
| | | GGTGGTTC | | AAGTG | | |
| | 3677 | GCTCCGACACCAT | 3678 | GCAACAGACGCAACC | ubiqui- | BT012698⁴ |
| | | TGACAAC | | AGAC | tin | |
| | 3685 | TTGCTTGGAGGA | 3686 | GCAAACAGAACCCCT | SAN | AK-247923³ |
| | | ACAGACG | | GAATC | D- | |
| | | | | | tinnily | |
| | | | | | protein | |

1. Vrebalov J, Ruezinsky D, Padmanabhan V, White R, Medrano D, Drake R, Schuch W, Giovannoni J. (2002) A MADS-box gene necessary for fruit ripening at the tomato ripening-inhibitor (rin) locus. Science 296: 343-346
2. Martel C, Vrebalov. J, Tafelmeyer P, Giovannoni J. (2011) The Tomato MADS-Box Transcription Factor RIPENING INHIBITOR Interacts with Promoters Involved in Numerous Ripening Processes in a COLERLESS NONRIPENING-Dependent Manner. Plant physiology 157: 1568-1579
3. Remans T, Smeets K, Opdenakker K, Cuypers A; Planta. 2008 Normalisation of real-time RT-PCR gene expression measurements in Arabidopsis thaliana exposed to increased metal concentrations. 227:1343-1349
4. Trond Løvdal, Cathrine Lillo (2009) Reference gene selection for quantitative real-time PCR normalization in tomato subjected to nitrogen, cold, and light stress. Analytical Biochemistry 387, 238-242

The probability associated with a Student's paired t-Test, with a two-tailed distribution for the data presented in each of FIG. 3A-H is given below:

### E4 (FIG. 3A)

| | 2558 BR | 5225 BR | 5996 BR |
|---|---|---|---|
| Wild type BR | <0.001 | <0.1 | n.s. |
| rin BR | <0.01 | <0.001 | <0.01 |

n.s. means not significant (P > 0.1)

### E8 (FIG. 3B)

| | 2558 BR | 5225 BR | 5996 BR |
|---|---|---|---|
| Wild type BR | <0.001 | <0.001 | <0.001 |
| rin BR | <0.01 | <0.001 | n.s. |

n.s. means not significant (P > 0.1)

### ACO1 (FIG. 3C)

| | 2558 BR | 5225 BR | 5996 BR |
|---|---|---|---|
| Wild type BR | <0.01 | <0.1 | <0.1 |
| rin BR | n.s. | <0.001 | <0.1 |

n.s. means not significant (P > 0.1)

### ACS2 (FIG. 3D)

| | 2558 BR | 5225 BR | 5996 BR |
|---|---|---|---|
| Wild type BR | <0.01 | n.s. | <0.1 |
| rin BR | <0.01 | <0.001 | <0.01 |

| | |
|---|---|
| n.s. means not significant (P > 0.1) | n.s. |

### ACS4 (FIG. 3E)

| | 2558 BR | 5225 BR | 5996 BR |
|---|---|---|---|
| Wild type BR | <0.1 | n.s. | <0.1 |
| rin BR | <0.01 | <0.001 | <0.1 |

n.s. means not significant (P > 0.1)

### Rin (FIG. 3F)

| | 2558 BR | 5225 BR | 5996 BR |
|---|---|---|---|
| Wild type BR | <0.1 | <0.1 | <0.1 |
| rin BR | — | — | — |

n.s. means not significant (P > 0.1);
— means RIN is not expressed

### MC (FIG. 3G)

| | 2558 BR | 5225 BR | 5996 BR |
|---|---|---|---|
| Wild type BR | <0.1 | <0.1 | n.s. |
| rin BR | — | — | — |

n.s. means not significant (P > 0.1);
— means RIN is not expressed

### Rin-MC (FIG. 3H)

Probability associated with a Student's paired t-Test, with a two-tailed distribution could not be determined as no protein was expressed in any of the mutants 2558, 5225 or 5996.

In Example 4 it is clearly shown that the reduced function rin protein according to the invention, as exemplified in mutants 2558, 5225 and 5996, are not loss-of-function rin proteins, as is described for the existing rin/rin mutant plants. It is known that existing rin/rin mutant plants have a deletion in their genomic DNA comprising part of the Rin and part of the MC sequence. This is confirmed in FIG. 3H which shows NRQ using the forward primer of RIN combined with the reversed primer for MC. With this particular combination of primers, only existing rin plants (rin) show a value (only this plant produces the fusion protein defined by this specific pair of primers), while Wild Type (WT) nor any of the mutants according to the invention do not, as expected.

In FIG. 3A it is shown that mutant 2558 differs from WT at the expression of E4 at Breaker Stage: NRQ WT(BR) is 1428 while NRQ 2558 (BR) is 112. The t-Test shows that the probability that the expression of E4 in WT(BR) is higher than in 2558 (BR) is >99.9%.

Also in FIG. 3B it is shown that the 3 mutants according to the invention differ from WT plants e.g when comparing NRQ of E8. The tTest shows that the probability that the expression of E8 in WT(BR) is higher than in 2558 (BR) or in 5225(BR) or in 5996(BR) is >99.9%.

The difference between the plants according to the invention and existing rin/rin mutant plants is shown for example in FIG. 3F. In FIG. 3F NRQ for the expression of Rin is shown. Existing rin/rin mutant plants (rin) do not express Rin at MG or BR stage, while plants of the invention do as illustrated. Also when the expression of MC is considered, like is illustrated in FIG. 3G, clear differences between existing rin/rin mutant plants (no expression of MC determined) and plants of the invention (significant higher, especially in BR stage) are observed.

This Example 4 thus clearly shows that plants of the invention relate to a cultivated plant of the species Solanum lycopersicum comprising a rin allele having one or more mutations, said mutations resulting in production of a mutant rin protein while existing rin/rin mutant plants do not produce Rin protein.

1. A cultivated plant of the species Solanum lycopersicum comprising a rin allele having one or more mutations, said mutations resulting in production of a mutant rin protein having reduced function compared to wild type Rin protein.

2. The cultivated plant according to claim 1 wherein said mutation or mutations result in delayed fruit ripening and/or a longer shelf life compared to Solanum lycopersicum being homozygous for the wild type Rin allele.

3. The cultivated plant according to claim 1 or 2 wherein said mutation or mutations result in the tomato fruits requiring significantly more days to reach the red stage compared to Solanum lycopersicum being homozygous for the wild type Rin allele.

4. The plant according to any one of claims 1 to 3 wherein the reduced function of the mutant rin protein is due to one or more amino acids being deleted, replaced and/or inserted compared to the wild type Rin protein of SEQ. ID NO: 1.

5. The plant according to any one of the preceding claims, wherein said mutant rin protein has a functional MADS-box domain.

6. The plant according to any one of the preceding claims, wherein said reduced function of the mutant rin protein is due to one or more amino acids being deleted, replaced and/or inserted in the K-domain.

7. The plant according to any one of the preceding claims, wherein the mutant rin protein has an amino acid sequence comprising at least 98% sequence identity to SEQ ID NO: 2, SEQ ID NO: 3 or SEQ ID NO: 4.

8. The plant according to any one of the preceding claims, wherein said mutant rin protein has one or more amino acids changed selected from the group consisting of Leu112Pro, Gly102Lys and the complete deletion of exon 3.

9. Seeds from which a plant according to any one of the preceding claims can be grown.

10. Tomato fruit, seeds, pollen, plant parts, and progeny of the plant of anyone of claims 1-9 comprising a rin allele having one or more mutations, said mutations resulting in production of a mutant rin protein having reduced activity compared to wild type Rin protein.

11. The tomato fruit of claim 10, wherein the tomato fruit has delayed ripening and/or an increased shelf life compared to fruits from Solanum lycopersicum plants being homozygous for the wild type Rin allele

12. The fruit according to 11, wherein the shelf life is at least 2 days longer than the shelf life of a tomato fruit being homozygous for the wild type Rin allele.

13. The plant according to claims 1 to 8, wherein the plant is an F1 hybrid plant.

14. Food or food products comprising or consisting of fruits or fruit parts of any one of claims 10 to 12.

15. A method for producing a hybrid Solanum lycopersicum plant, said method comprising:
(a) obtaining a first Solanum lycopersicum plant of any one of claims 1-8 or a seed according to claim 9; and
(b) crossing said first Solanum lycopersicum plant with a second Solanum lycopersicum plant to obtain hybrid seeds;
wherein said hybrid Solanum lycopersicum plant grown from said hybrid seeds comprises a rin allele having one or more mutations wherein said mutations result in production of a mutant rin protein having reduced activity compared to wild type Rin protein.
